(19) United States
(12) Patent Application Publication
(10) Pub. No.: US 2013/0095503 A1
(43) Pub. Date: Apr. 18, 2013

(54) SERUM SPLA2-IIA AS DIAGNOSIS MARKER FOR PROSTATE AND LUNG CANCER

(71) Applicant: University of Cincinnati, Cincinnati, OH (US)

(72) Inventor: Shan Lu, Cincinnati, OH (US)

(73) Assignee: UNIVERSITY OF CINCINNATI, Cincinnati, OH (US)

(21) Appl. No.: 13/707,245

(22) Filed: Dec. 6, 2012

Related U.S. Application Data
(63) Continuation-in-part of application No. 13/520,586, filed on Jul. 5, 2012.
(60) Provisional application No. 61/567,460, filed on Dec. 6, 2011.
(60) Provisional application No. 61/292,270, filed on Jan. 5, 2010.
(60) Provisional application No. 61/400,606, filed on Jul. 30, 2010.
(60) Provisional application No. 61/400,806, filed on Aug. 3, 2010.

(51) Int. Cl. G01N 33/574

(52) U.S. Cl. CPC G01N 33/57488
USPC 435/7.4

(57) ABSTRACT

Kits for assessing lung cancer in patients with solitary pulmonary nodules and methods for assessing lung cancer. The kit includes reagents for detection and/or quantification of serum secretory phospholipase A₂-IIA in plasma, reagents for detection and/or quantification of carcinoembryonic antigen in plasma, and reagents for detection and/or quantification of cytokeratin-19 fragment in plasma. The method includes contacting a sample with a specific binding agent for serum secretory phospholipase A₂-IIA, a specific binding agent for carcinoembryonic antigen, and a specific binding agent for cytokeratin-19 fragment, calculating levels of serum secretory phospholipase A₂-IIA, carcinoembryonic antigen, and cytokeratin-19 fragment, and assessing as indicating lung cancer in the patient if the calculated levels are elevated.

This application is filed under 35 U.S.C. §111(a) as a continuation-in-part of U.S. application Ser. No. 13/520,586, filed on Jul. 5, 2012, which is a national stage entry of PCT/US2011/020225, filed on Jan. 5, 2011, which claims priority under 35 U.S.C. §119 to U.S. Provisional Application Ser. No. 61/292,270, filed Jan. 5, 2010, U.S. Provisional Application Ser. No. 61/400,606, filed Jul. 30, 2010, and U.S. Provisional Application Ser. No. 61/400,806, filed Aug. 3, 2010, the contents of which are hereby incorporated by reference in their entirety. This application also claims priority under 35 U.S.C. §119 to U.S. Provisional Application Ser. No. 61/567,460, filed on Dec. 6, 2011, the contents of which are hereby incorporated by reference in their entirety.

### TECHNICAL FIELD

The present disclosure relates to kits and methods for assessing lung cancer.

### BACKGROUND

It is widely accepted that many cancers arise from chronic inflammation. Chronic inflammation is a pathological condition characterized by concurrent active inflammation, tissue destruction, and attempted repair. Chronic inflammation results in a sustained innate immune response which creates a microenvironment rich in cytokines, chemokines, growth factors, and angiogenesis factors, and fosters cell proliferation and survival, a critical step in carcinogenesis. The nuclear factor-κB (hereinafter “NF-κB”) is a key linking molecule in inflammation and immunity to cancer development and progression. The NF-κB target genes, such as cyclooxygenase-2 (hereinafter “COX2”), matrix metalloproteinase (hereinafter “MMP”), VEGF, IL6, and IL8, also play a critical role in cell proliferation, angiogenesis, metastasis, and inflammation. Various carcinogens, oncogenes, and cell signaling pathways, such as EGFR-HER2-PI3K-Akt, activate NF-κB. Activation of NF-κB leads to expression of inflammatory cytokines and growth factors, blockade of apoptosis, promotion of proliferation, angiogenesis, and tumor invasion.

Prostate cancer and benign prostatic hyperplasia (hereinafter “BPH”) are two common male urinary diseases, which are often associated with overlapping signs and symptoms. BPH, a treatable disease, is a nonmalignant enlargement of the prostate; in contrast, cancer of the prostate is the second leading cause of cancer death among men in the United States. Standard diagnostic tests for prostate cancer include prostate specific antigen (hereinafter “PSA”), histopathology, Gleason score, and magnetic resonance imaging (hereinafter “MRI”). However, these diagnostic tests are limited; for example, PSA tests lack sensitivity (21%) with 91% specificity and PSA tests have not been validated in prostate cancer surveillance trials. Additionally, biopsies are prone to sampling errors and MRI can miss small tumors.

With particular regard to PSA, urologic guidelines have established a PSA value of 4.0 ng/mL as an upper limit of normal prostate cells. Accordingly, PSA levels of greater than 4.0 ng/mL typically mandate the performance of a biopsy to identify prostate cancer. However, plasma PSA levels may be elevated in benign diseases, such as in about 30-50% of benign prostate hyperplasia (i.e., BPH), leading to low specificity of PSA testing. Accordingly, it is estimated that greater than approximately 500,000 men will be subjected to unnecessary biopsies each year. Additionally, it is reported that prostate cancer is over-diagnosed by approximately 20-66% in the United States and in Europe. Moreover, many aggressive prostate cancers having high Gleason scores but low PSA fail to be detected via PSA testing, leading to low sensitivity. By way of example, the rate of prostate cancer detection is approximately 40%, 30%, 27%, 22%, and 19% corresponding to PSA levels of >10, >4-10, >2.5-4, >2-2.5, and ≦2 ng/mL. To date, biopsy-dependent Gleason scores remain the sole diagnostic modality with confirmed prognostic power.

The low mortality rate from prostate cancer after 15 years from diagnosis does not support aggressive treatment for indolent (i.e., insignificant or favorable) prostate cancers. As a result, patients with indolent prostate cancers are placed under active surveillance; however, progression and metastasis may eventually occur in some patients and PSA testing has not been validated for use in active surveillance trials.

Lung cancer is the most common cancer worldwide in both incidence and mortality; for example, approximately 1.3 million new cases of lung cancer are diagnosed each year and approximately 1.2 million deaths result from lung cancer each year, representing 30% of total cancer deaths. In the United States, lung cancer is the leading cause of cancer death; for example, over 220,000 new cases of lung cancer were diagnosed in the United States in 2011 and over 155,000 deaths resulted from lung cancer in the United States in 2011. Additionally, lung cancer has a much lower survival rate when compared to other common cancers; for example, there is only a 16% 5-year survival rate for lung cancer in the United States and a 10.9% 5-year survival rate for lung cancer in Europe. This is partly due to the fact that over 50% of patients receive late diagnoses of locally-advanced or metastatic disease. However, the 5-year survival rate for stage 1A non-small cell lung cancer (i.e., NSCLC) is as high as 73%, indicating that early diagnosis may enhance lung cancer survival.

Despite advances in diagnosis, treatment, and patient care, long-term survival rates of lung cancer patients have not improved significantly over the past 20 years. Currently, standard diagnostic tests for lung cancer include computed tomography (i.e., CT) including low dose spiral CT (i.e., LDCT), chest radiographs (i.e., CXRs), and sputum cytology. While increased sensitivity of imaging technology in LDCT has allowed for the detection of lung cancer at an earlier stage, LDCT is limited in its inability to distinguish malignant nodules from benign tumors and/or inflammatory pseudo tumors.

For example, the presence of solitary pulmonary nodules (hereinafter, “SPNs”), which are less than 3 cm in diameter, is commonly encountered in clinical practice, wherein the presence of SPNs is about 10-20% in the United States in baseline screening. However, histoplasmosis is an epidemic in the Ohio valley which leads to lung nodule rates as high as 61%. The National Lung Screening Trial (i.e., NLST) demonstrated a 20% reduction in lung cancer death using LDCT relative to CXRs; however, approximately 25% of the candidates in such trial had SPNs among which the rate of lung cancer was only about 3.6%. While some SPNs are malignant, others may be indicative of slowly growing indolent lung cancer, benign tumors, and/or inflammatory pseudo tumors. The identification of malignant SPNs is crucial because SPNs represent a localized and potentially curable form of lung cancer. However, such identification of malignant SPNs is challenging; currently, monitoring of SPNs requires repeated CT scans at 3, 6, 9, 12, and 24 months based upon the size of the SPNs, potentially resulting in radiation exposure and anxiety in patients.

Patients may also undergo invasive procedures for lung cancer diagnosis such as bronchoscopy, thoracoscopy, thoracotomy, and CT-guided fine needle aspiration, potentially resulting in complications in the patients such as lung collapse and death. Additionally, biopsies for lung cancer diagnosis and histological subtyping cannot be performed for many patients due to multimorbidity, unstable clinical conditions, and/or unfavorable tumor localization. While a few serum biomarkers for lung cancer are currently under investigation, such as carcinoembryonic antigen (i.e., CEA) and cytokeratin-19 fragment (i.e., Cyfra 21.1) for NSCLC and neuron-specific enolase (i.e., NSE) for small cell lung cancer (i.e., SCLC), such biomarkers are lacking in sensitivity and specificity for predicting early stage lung cancers.

### SUMMARY

The present disclosure is based on the discovery that serum secretory phospholipase A₂-IIA, (hereinafter “serum sPLA2-IIA”), is a serum diagnosis marker for prostate and/or lung cancer. sPLA2-IIA is both a target and effector gene of NF-κB. Moreover, sPLA2-IIA is a secretory phospholipid hydrolase that mediates the release of arachidonic acid and lysophosphatidylcholine. Accordingly, in one embodiment, a method for diagnosing prostate cancer in a subject is disclosed.

In one embodiment, a kit for assessing lung cancer in patients with solitary pulmonary nodules is disclosed. The kit includes reagents for detection and/or quantification of serum secretory phospholipase A₂-IIA in plasma, reagents for detection and/or quantification of carcinoembryonic antigen in plasma, and reagents for detection and/or quantification of cytokeratin-19 fragment in plasma.

In another embodiment, a kit for assessing lung cancer in patients with solitary pulmonary nodules is disclosed. The kit includes reagents for detection and/or quantification of serum secretory phospholipase A₂-IIA in plasma, wherein the reagents for detection and/or quantification of serum secretory phospholipase A₂-IIA in plasma include a capture antibody having specific binding affinity to the serum secretory phospholipase A₂-IIA in the plasma, an enzyme conjugated to a detection antibody having specific binding affinity to the serum secretory phospholipase A₂-IIA in the plasma, and a substrate of the enzyme conjugated to the detection antibody having specific binding affinity to the serum secretory phospholipase A₂-IIA in the plasma, wherein action of the enzyme conjugated to the antibody having specific binding affinity to the serum secretory phospholipase A₂-IIA in the plasma upon the substrate produces at least one detectable product. The kit also includes reagents for detection and/or quantification of carcinoembryonic antigen in plasma, wherein the reagents for detection and/or quantification of carcinoembryonic antigen in plasma include a capture antibody having specific binding affinity to the carcinoembryonic antigen in the plasma, an enzyme conjugated to a detection antibody having specific binding affinity to the carcinoembryonic antigen in the plasma, and a substrate of the enzyme conjugated to the detection antibody having specific binding affinity to the carcinoembryonic antigen in the plasma, wherein action of the enzyme conjugated to the detection antibody having specific binding affinity to the carcinoembryonic antigen in the plasma upon the substrate produces at least one detectable product. The kit also includes reagents for detection and/or quantification of cytokeratin-19 fragment in plasma, wherein the reagents for detection and/or quantification of cytokeratin-19 fragment in plasma includes a capture antibody having specific binding affinity to the cytokeratin-19 fragment in the plasma, an enzyme conjugated to a detection antibody having specific binding affinity to the cytokeratin-19 fragment in the plasma, and a substrate of the enzyme conjugated to the detection antibody having specific binding affinity to the cytokeratin-19 fragment in the plasma, wherein action of the enzyme conjugated to the detection antibody having specific binding affinity to the cytokeratin-19 fragment in the plasma upon the substrate produces at least one detectable product.

In yet another embodiment, an in vitro method for assessing lung cancer in a patient is disclosed. The method includes: (a) contacting a portion of a sample from the patient with a specific binding agent for serum secretory phospholipase A₂-IIA, a specific binding agent for carcinoembryonic antigen, and a specific binding agent for cytokeratin-19 fragment, (b) calculating levels of serum secretory phospholipase A₂-IIA, carcinoembryonic antigen, and cytokeratin-19 fragment based on the contacting in step (a), and (c) assessing as indicating lung cancer in the patient if the calculated levels of serum secretory phospholipase A₂-IIA, carcinoembryonic antigen, and/or cytokeratin-19 fragment are elevated relative to cutoff values of serum secretory phospholipase A₂-IIA, carcinoembryonic antigen, and cytokeratin-19.

Additional features and advantages of the embodiments described herein will be set forth in the detailed description which follows, and in part will be readily apparent to those skilled in the art from that description or recognized by practicing the embodiments described herein, including the detailed description which follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

The following detailed description of the embodiments of the present disclosure can be better understood when read in conjunction with the following drawings, where like structure is indicated with like reference numerals, and in which:

FIG. 1 is a bar graph of serum sPLA2-IIA(-800)-Luc (0.25 μg/well) transfected LNCaP-AI cells (10⁵ cells/well in 12-well plate) and serum sPLA2-IIA(-800)-Luc (0.25 μg/well) transfected LNCap-AI cells (10⁵ cells/well in 12-well plate) treated with epidermal growth factor (100 ng/mL) without or with Erlotinib (˜20 μM), Gefitinib (˜20 μM), Lapatinib (˜20 μM), CI-1033 (˜8 μM), LY294002 (˜20 μM), and Bortezomib (˜20 μM) with respect to luciferase activity (×10⁻⁷, Light units/mg protein);

FIG. 2 is a western blot which depicts the expression of serum sPLA2-IIA protein in LNCaP-AI cells treated with Erlotinib (˜20 μM), Gefitinib (˜20 μM), Lapatinib (˜20 μM), CI-1033 (˜8 μM), and LY294002 (˜20 μM) without or with EGF (˜100 ng/mL);

FIG. 3 is a western blot which depicts the expression of serum sPLA2-IIA protein in LNCaP-AI cells treated with Bortezomib (˜20 μM) with or without EGF (˜100 ng/mL);

FIG. 4 is a western blot which depicts the expression of serum sPLA2-IIA protein in LNCaP-AI cells treated with Erlotinib (˜20 μM), Gefitinib (˜20 μM), Lapatinib (˜20 μM), CI-1033 (˜8 μM), and LY294002 (˜20 μM);

FIG. 5 is a western blot which depicts the expression of serum sPLA2-IIA protein in LNCaP-AI cells treated with Lapatinib (˜20 μM);

FIG. 6 is a western blot which depicts the expression of serum sPLA2-IIA protein in LNCaP-AI cells treated with Heregulin-α (˜50 ng/mL);

FIG. 7 is a bar graph of serum sPLA2-IIA (ng/mL) in LNCaP-AI cells treated with Erlotinib (˜20 μM), Gefitinib (˜20 μM), Lapatinib (˜20 μM), CI-1033 (˜8 μM), LY294002 (˜20 μM), and Bortezomib (˜20 μM);

FIG. 8 is a bar graph of mRNA expression levels of serum sPLA2-IIA in LNCaP and LNCaP-AI cells;

FIG. 9 is a western blot which depicts the expression of serum sPLA2-IIA protein;

FIG. 10 is a bar graph of sPLA2-IIA (ng/mL) in the conditioned medium secreted by LNCaP-AI (500,000 cells/well in 6 well plate) and LNCaP cells (500,000 cells/well in 6 well plate) by ELISA assay;

FIG. 11 is a graph of LNCaP-AI cells cultured in 10% stripped medium in the presence of EGF (ng/mL) or serum sPLA2-IIA (ng/mL) for about 4 days with respect to optical density (570 nM);

FIG. 12 is a graph of LNCaP cells cultured in 10% stripped medium in the presence of cFLSYR (μM) or c(2NapA)LS(2NapA)R (μM) for about 4 days with respect to optical density (570 nM);

FIG. 13 is a graph of plasma samples from healthy donors (20 samples) and prostate cancer patients (43 samples) with respect to the level of serum sPLA2-IIA (pg/mL);

FIG. 14 is an immunohistochemistry stain of (A) a lesion of Gleason score 6, (B) a lesion of Gleason score 7, (C) a lesion of Gleason score 8, and (D) benign prostate hyperplasia, wherein solid arrows indicate benign prostatic glands which are negative and serve as controls and open arrows indicate prostate cancer cells;

FIG. 15 is a graph of plasma samples from healthy donors (20 samples) and lung cancer patients (10 samples) with respect to the level of serum sPLA2-IIA (pg/mL);

FIG. 16 is a graph of plasma samples from healthy donors, heavy smokers, and lung cancer patients with respect to the level of serum sPLA2-IIA (pg/mL);

FIG. 17 is a graph of plasma samples from benign nodules and lung cancer patients with respect to the level of serum sPLA2-IIA (pg/mL);

FIG. 18 is a graph of plasma samples from lung cancer patients with stage two and stage three cancer relative to early stage one cancer with respect to the level of sPLA2-IIA (pg/mL);

FIG. 19 is a graph of (A) plasma samples from patients having benign SPNs and lung cancer with respect to plasma sPLA2-IIA (pg/mL) with cutoff value (dotted line), and (B) plasma samples from patients having stage one and stage two lung cancer with respect to plasma sPLA2-IIA (pg/mL) with cutoff value (dotted line);

FIG. 20 is a graph of (A) specificity (100%-Specificity %) of 96 lung cancer specimens from benign nodule-lung cancer cohort versus 20 specimens from healthy donors with respect to sensitivity (%), (B) specificity (100%-Specificity %) of 96 lung cancer specimens from BNLCC relative to 29 benign SPN specimens from BNLCC with respect to sensitivity (%), and (C) specificity (100%-Specificity %) of 18 stage 2 lung cancer specimens from BNLCC relative to 29 benign SPN specimens from BNLCC;

FIG. 21 is an immunohistochemistry stain of serum sPLA2-IIA expression in lung cancer specimens in (A) squamous cell carcinoma, (B) adenocarcinoma, (C), small cell carcinoma, (D) bronchioalveolar carcinoma, (E) metastatic squamous cell carcinoma with necrosis, (F) atypical carcinoid, (G) inflammatory pseudotumor, (H) normal lung tissue, and (I) SP-C/Tag mouse lung cancer;

FIG. 22 is a western blot which depicts (A) the expression of P-HER2, HER2, and β-actin in A549 cells treated with sPLA2-IIA (0, 0.125, 0.25, or 0.5 μg/mL), and (B) the expression of P-HER3, HER3, and β-actin in H1975 cells treated with sPLA2-IIA (0, 0.125, 0.25, or 0.5 μg/mL);

FIG. 23 is a western blot which depicts (A) the expression of P-HER2, HER2, and β-actin in LNCaP-A1 cells treated with sPLA2-IIA (0.5 μg/mL) for 0 to 180 minutes (0, 30, 60, 120, or 180 min), and (B) the expression of P-HER2, HER2, and β-actin in LNCaP-A1 cells treated with sPLA2-IIA (0, 0.125, 0.25, or 0.5 μg/mL);

FIG. 24 is a graph of plasma samples from patients having stage T1-T4 prostate cancer with respect to plasma sPLA2-IIA (pg/mL) with cutoff value (dotted line) and mean values (solid lines); and

FIG. 25 is a graph of (A) specificity (100%-Specificity %) of eighteen specimens of stage T1 prostate cancer versus 101 specimens of advanced stage T2-T4 prostate cancer with respect to sensitivity (%), and (B) specificity (100%-Specificity %) of 85 specimens of Gleason scores 6-7 prostate cancer versus 41 specimens of Gleason scores 8-10 prostate cancer with respect to sensitivity (%) wherein AUC and 95% confidence interval (hereinafter “CI”) were determined.

Skilled artisans appreciate that elements in the figures are illustrated for simplicity and clarity and are not necessarily drawn to scale. For example, the dimensions of some of the elements in the figures may be exaggerated relative to other elements, as well as conventional parts removed, to help to improve understanding of the various embodiments of the present disclosure.

### DETAILED DESCRIPTION

The following terms are used in the present application:

As used herein, the terms “diagnosing”, “diagnosed”, and “diagnose” refer to determining the presence and/or absence of a disease or condition based upon an evaluation of physical signs, symptoms, history, laboratory test results, and/or procedures. Specifically, in the context of prostate cancer and/or lung cancer, diagnosing refers to determining the presence or absence of a disease or condition based upon an evaluation of the level of serum sPLA2-IIA, carcinoembryonic antigen (i.e., CEA), cytokeratin-19 fragment (i.e., Cyfra 21.1), squamous cell carcinoma antigen (i.e., SCCA), neuron specific enolase (i.e., NSE), and progastrin releasing peptide (i.e., ProGRP).

As used herein, the term “positive diagnosis” refers to a determination of the presence of a disease or condition based upon an evaluation of physical signs, symptoms, history, laboratory test results, and/or procedures. In the context of prostate cancer, a positive diagnosis refers to a determination of the presence of prostate cancer based upon an evaluation of the level of serum sPLA2-IIA. Similarly, in the context of lung cancer, a positive diagnosis refers to a determination of the presence of lung cancer based upon an evaluation of the level of serum sPLA2-IIA, CEA, Cyfra 21.1, SCCA, NSE, and/or ProGRP.

As used herein, the term “negative diagnosis” refers to a determination of the absence of a disease or condition based upon an evaluation of physical signs, symptoms, history, laboratory test results, and/or procedures. In the context of prostate cancer, a negative diagnosis refers to a determination of the absence of prostate cancer based upon an evaluation of the level of serum sPLA2-IIA. Similarly, in the context of lung cancer, a negative diagnosis refers to a determination of the absence of lung cancer based upon an evaluation of the level of serum sPLA2-IIA, CEA, Cyfra 21.1, SCCA, NSE, and/or ProGRP.

In the context of serum sPLA2-IIA, the term “elevated level” refers to the level of serum sPLA2-IIA in a biological sample which is greater than a baseline level of serum sPLA2-IIA. For example, in the context of prostate cancer, an elevated level of serum sPLA2-IIA in blood plasma is from about 400 pg/mL to about 18,000 pg/mL, or from about 400 pg/mL to about 7,300 pg/mL, or from about 500 pg/mL to about 7,300 pg/mL, or from about 1,100 pg/mL to about 18,000 pg/mL. In the context of lung cancer, an elevated level of serum sPLA2-IIA in blood plasma is from about 400 pg/mL to about 16,000 pg/mL, or from about 400 pg/mL to about 7,500 pg/mL, or from about 1,200 pg/mL to about 15,000 pg/mL. In one embodiment, the elevation of the level of serum sPLA2-IIA in the biological sample is statistically significant.

Similarly, in the context of prostate specific antigen, the term “elevated level” refers to the level of prostate specific antigen in a biological sample which is greater than a baseline level of prostate specific antigen. For example, an elevated level of prostate specific antigen is from about 4 ng/mL to about 1,600 ng/mL.

As used herein, the term “baseline level” refers to the level of serum sPLA2-IIA in a biological sample from a subject who is not suffering from prostate cancer and/or lung cancer. In the context of prostate cancer, baseline level refers to the level of serum sPLA2-IIA in subjects with normal prostate tissue and/or in subjects with benign prostate disease. For example, in the context of prostate cancer, a baseline level of serum secretory sPLA2-IIA in blood plasma is from about 0 pg/mL to about 2,000 pg/mL. In the context of lung cancer, baseline level refers to the level of serum sPLA2-IIA in subjects with normal lung tissue, in subjects with benign lung diseases, and/or in subjects with benign solitary pulmonary nodules. For example, in the context of lung cancer, a baseline level of serum sPLA2-IIA in blood plasma is from about 0 pg/mL to about 2,400 pg/mL.

Similarly, in the context of prostate specific antigen, the term “baseline level” refers to the level of prostate specific antigen in a biological sample from a subject who is not suffering from prostate cancer. For example, a baseline level of prostate specific antigen is from about 0 ng/mL to about 4 ng/mL.

As used herein, the term “Gleason score” refers to system for scoring and/or measuring the aggressiveness of prostate cancer determined from tissue samples taken during a biopsy. A Gleason score may be used to help evaluate the prognosis of men with prostate cancer. Gleason scores range from about 6 to about 10. Generally, prostate cancers with higher Gleason scores are more aggressive.

As used herein, the term “cutoff value” refers to a threshold value which distinguishes subjects suffering from a disease or condition from subjects who are not suffering from the disease or condition. In the context of prostate cancer and lung cancer, an elevated level of serum sPLA2-IIA is greater than the cutoff value and a non-elevated level of serum sPLA2-IIA is less than the cutoff value. Specifically regarding prostate cancer, the cutoff value of serum sPLA2-IIA is about 2 ng/mL. Specifically regarding lung cancer, the cutoff value of serum sPLA2-IIA is about 2.0 ng/mL, or more particularly, about 2.4 ng/mL. Also regarding lung cancer, the cutoff value of CEA is about 6.0 ng/mL, the cutoff value of Cyfra 21.1 is 3.3 ng/mL, the cutoff value of SCCA is 2.0 ng/mL, the cutoff value of NSE is 12.5 ng/mL, and the cutoff value of ProGRP is 300 pg/mL.

### I. Diagnosing Prostate Cancer

Embodiments of the present disclosure relate to methods for diagnosing prostate cancer and lung cancer in a subject. In one embodiment, a method for diagnosing prostate cancer in a subject is disclosed. In one particular embodiment, a method for diagnosing prostate cancer in a subject is disclosed, wherein the method includes: (a) obtaining a biological sample from the subject; (b) determining a level of serum sPLA2-IIA in the biological sample; (c) comparing the level of serum sPLA2-IIA determined in step (b) with a baseline level of serum sPLA2-IIA; and (d) diagnosing prostate cancer in the subject, wherein an elevated level of serum sPLA2-IIA as compared to the baseline level correlates to a positive diagnosis of prostate cancer in the subject.

In one embodiment, the method for diagnosing prostate cancer in the subject includes obtaining the biological sample from the subject in step (a). In one particular embodiment, the biological sample is blood. In a further embodiment, the biological sample is at least one of plasma and/or serum. In still a further embodiment, the biological sample is plasma. In another embodiment, the subject is human. Accordingly, obtaining the biological sample from the subject in step (a) of the method for diagnosing prostate cancer may include blood testing. In contrast to biopsies, blood testing is minimally invasive. Blood testing may be performed according to any blood testing methods known in the field. For example, in one particular embodiment, blood testing may be performed by extracting blood from the subject with a needle via venipuncture.

In yet another embodiment, the method for diagnosing prostate cancer in the subject includes determining a level of serum sPLA2-IIA in the biological sample in step (b). In one embodiment, determining the level of serum sPLA2-IIA in the biological sample includes performing an in vitro assay. The in vitro assay may be selected from the group consisting of immunoassays, aptamer-based assays, histological assays, cytological assays, and mRNA expression level assays. The in vitro assay should not be limited to those disclosed herein, however, but may be performed according to any methods known in the fields of biochemistry, molecular biology, and/or medical diagnostics. In one particular embodiment, the in vitro assay is an immunoassay, and more particularly, an enzyme-linked immunosorbent assay.

In another embodiment, the method for diagnosing prostate cancer in the subject includes comparing the level of serum sPLA2-IIA previously determined with a baseline level of serum sPLA2-IIA in step (c). The baseline level of serum sPLA2-IIA in step (c) may be determined in subjects with normal prostate tissue and/or in subjects with benign prostate disease. In one particular embodiment, the baseline level of serum sPLA2-IIA functions as a control for the method.

In yet another embodiment, the method for diagnosing prostate cancer in the subject includes diagnosing prostate cancer in the subject in step (d), wherein an elevated level of serum sPLA2-IIA as compared to the baseline level correlates to a positive diagnosis of prostate cancer in the subject. In one particular embodiment, the elevated level of serum sPLA2-IIA is from about 400 pg/mL to about 18,000 pg/mL. In this particular embodiment, the biological sample has a Gleason score of from about 6 to about 10. In a further embodiment, the biological sample has a Gleason score of from about 8 to about 10. In another embodiment, the elevated level of serum sPLA2-IIA is from about 400 pg/mL to about 7,300 pg/mL. In this particular embodiment, the biological sample has a Gleason score of from about 6 to about 7.

In one particular embodiment, the elevated level of serum sPLA2-IIA correlates to a positive diagnosis of prostate cancer in the subject independent of the level of prostate specific antigen in the biological sample. Alternatively, in another embodiment, the elevated level of serum sPLA2-IIA correlates to a positive diagnosis of prostate cancer in the subject in conjunction with the level of prostate specific antigen in the sample. In this particular embodiment, the method for diagnosing prostate cancer in the subject further includes determining a level of prostate specific antigen in the biological sample and comparing the level of prostate specific antigen with a baseline level of prostate specific antigen. In this embodiment, an elevated level of prostate specific antigen as compared to the baseline level correlates to a positive diagnosis of prostate cancer. In one embodiment, the level of prostate specific antigen is determined via in vitro assay as previously described above. In one particular embodiment, the in vitro assay is an immunoassay, and more particularly, is an enzyme-linked immunosorbent assay.

In another embodiment, the elevated level of serum sPLA2-IIA increases with the progression (i.e. increasing severity) of the prostate cancer. Prostate cancer increases in severity in the order of stages. For example, in stage two prostate cancer, the elevated level of serum sPLA2-IIA in the biological sample is from about 500 pg/mL to about 7,300 pg/mL. Additionally, as another example, in stage three prostate cancer, the elevated level of serum sPLA2-IIA in the biological sample is from about 1,100 pg/mL to about 18,000 pg/mL.

Accordingly, in one embodiment, the method for diagnosing prostate cancer in the subject further includes determining a stage of the prostate cancer, wherein an elevated level of serum sPLA2-IIA in the biological sample of from about 500 pg/mL to about 7,300 pg/mL correlates to a diagnosis of stage two prostate cancer in the subject. In another embodiment, an elevated level of serum sPLA2-IIA in the biological sample of from about 1,100 pg/mL to about 18,000 pg/mL correlates to a diagnosis of stage three prostate cancer in the subject.

In still another embodiment, a non-elevated level of serum sPLA2-IIA in the biological sample of serum sPLA2-IIA as compared to the baseline level correlates to a negative diagnosis of prostate cancer in the subject. In one particular embodiment, the non-elevated level of serum sPLA2-IIA as compared to the baseline level correlates to a diagnosis of normal prostate tissue and/or benign prostatic diseases. In a further embodiment, the benign prostatic disease is benign prostatic hyperplasia.

In yet another embodiment, the method for diagnosing prostate cancer in the subject further includes determining a cutoff value, wherein the non-elevated level of serum sPLA2-IIA is less than the cutoff value. In one particular embodiment, the cutoff value is about 2 ng/mL.

Embodiments of methods of diagnosing prostate cancer have now been described in detail. Further embodiments directed to managing treatment of prostate cancer will now be described.

### II. Managing Treatment of Prostate Cancer

In another embodiment, a method for managing treatment of a patient suspected of having indolent prostate cancer is disclosed. The method includes (a) assessing prostate cancer in vitro in the patient by calculating a level of sPLA2-IIA. An elevated level of sPLA2-IIA relative to a cutoff value of sPLA2-IIA is indicative of prostate cancer. The method also includes treating the patient for prostate cancer where prostate cancer is indicated in step (a), where the calculated level of sPLA2-IIA is elevated. The method also includes placing the patient under active surveillance where prostate cancer is not indicated in step (a), wherein the calculated level of sPLA2-IIA is not elevated. The level of sPLA2-IIA may be calculated as set forth in the Instructions for Utilizing the Kit for Assessing Lung Cancer as described in greater detail in a later section. In one embodiment, the cutoff value for sPLA2-IIA is 2.0 ng/mL.

In one embodiment, the method further includes treating the patient for prostate cancer with Lapatinib and/or Bortezomib where the patient has a high level of sPLA2-IIA. In this particular embodiment, a high level of sPLA2-IIA corresponds to an aggressive cancer. In another embodiment, the method further includes monitoring the patient suspected of having indolent prostate cancer by serially calculating the level of sPLA2-IIA in the sample, where prostate cancer is not indicated in step (a).

In another embodiment, a method of managing treatment of a patient suspected of having prostate cancer is disclosed. In one embodiment, the method includes (a) assessing prostate cancer in vitro in the patient by calculating in a plasma sample from the patient a level of serum secretory phospholipase A₂-IIA, wherein an elevated level of serum secretory phospholipase A₂-IIA relative to a cutoff value of serum secretory phospholipase A₂-IIA is indicative of prostate cancer. The method may also include (b) treating the patient for prostate cancer where prostate cancer is indicated, wherein the calculated level of serum secretory phospholipase A₂-IIA in step (a) is a baseline level. Additionally, the method may also include (c) determining treatment response of the patient by calculating in a sample from the patient a post-treatment level of serum secretory phospholipase A₂-IIA, wherein a decreased post-treatment level compared to the baseline level is indicative of a successful treatment response. In one embodiment, the treatment includes surgical resection, chemotherapy, radiation therapy, biological therapy, and combinations thereof.

In another embodiment, a method of managing treatment in a patient suffering from prostate cancer is disclosed. The method includes (a) establishing a baseline level of sPLA2-IIA in a plasma sample from the patient; (b) initiating treatment; and (c) serially monitoring the level of sPLA2-IIA in the patient during treatment, wherein the treatment is managed to decrease the level of sPLA2-IIA in the patient.

Embodiments of managing treatment of prostate cancer have now been described in detail. Further embodiments directed to diagnosing lung cancer will now be described.

### III. Diagnosing Lung Cancer

In another embodiment, a method for diagnosing lung cancer in a subject is disclosed, wherein the method includes: (a) obtaining a biological sample from the subject; (b) determining a level of serum sPLA2-IIA in the biological sample; (c) comparing the level of serum sPLA2-IIA determined in step (b) with a baseline level of serum sPLA2-IIA; and (d) diagnosing lung cancer in the subject, wherein an elevated level of serum sPLA2-IIA as compared to the baseline level correlates to a positive diagnosis of lung cancer in the subject.

In one embodiment, the method for diagnosing lung cancer in the subject includes obtaining the biological sample from the subject in step (a). In one particular embodiment, the biological sample is blood. In a further embodiment, the biological sample is at least one of plasma and/or serum. In still a further embodiment, the biological sample is plasma. In another embodiment, the subject is human. Accordingly, obtaining the biological sample from the subject in step (a) of the method for diagnosing lung cancer may include blood testing as previously described above.

In another embodiment, the method for diagnosing lung cancer in the subject includes determining a level of serum sPLA2-IIA in the biological sample in step (b). In one embodiment, determining the level of serum sPLA2-IIA in the biological sample includes performing an in vitro assay. The in vitro assay may be performed as previously described above. In one particular embodiment, the in vitro assay is an immunoassay, and more particularly is an enzyme-linked immunosorbent assay.

In another embodiment, the method for diagnosing lung cancer in the subject includes comparing the level of serum sPLA2-IIA previously determined with a baseline level of serum sPLA2-IIA in step (c). The baseline level of serum sPLA2-IIA in step (c) may be determined in subjects with normal lung tissue and/or in subjects with benign lung diseases. In one particular embodiment, the baseline level of serum sPLA2-IIA functions as a control for the method.

In yet another embodiment, the method for diagnosing lung cancer in the subject includes diagnosing lung cancer in the subject in step (d), wherein an elevated level of serum sPLA2-IIA as compared to the baseline level correlates to a positive diagnosis of lung cancer in the subject. In one particular embodiment, the elevated level of serum sPLA2-IIA is from about 400 pg/mL to about 16,000 pg/mL. In this particular embodiment, the elevated level of sPLA2-IIA correlates to a positive diagnosis of lung cancer. The lung cancer is selected from the group consisting of non-small cell lung cancer, small cell carcinoma, and metastatic squamous cell carcinoma. In a further aspect, the non-small cell lung cancer is selected from the group consisting of squamous cell carcinoma, adenocarcinoma, and bronchioalveolar carcinoma.

In another embodiment, the elevated level of serum sPLA2-IIA increases with the progression (i.e. increasing severity) of the lung cancer. Lung cancer increases in severity in the order of stages. For example, in stage one lung cancer, the elevated level of serum sPLA2-IIA in the biological sample is from about 400 pg/mL to about 7,500 pg/mL. Additionally, as another example, in stage two or stage three lung cancer, the elevated level of serum sPLA2-IIA in the biological sample is from about 1,200 pg/mL to about 15,000 pg/mL.

Accordingly, in one embodiment, the method for diagnosing lung cancer in the subject further includes determining a stage of the lung cancer, wherein an elevated level of serum sPLA2-IIA in the biological sample of from about 400 pg/mL to about 7,500 pg/mL correlates to a diagnosis of stage one lung cancer in the subject. In another embodiment, an elevated level of serum sPLA2-IIA in the biological sample of from about 1,200 pg/mL to about 15,000 pg/mL correlates to a diagnosis of stage two or stage three lung cancer in the subject.

In still another embodiment, a non-elevated level of serum sPLA2-IIA in the biological sample of serum sPLA2-IIA as compared to the baseline level correlates to a negative diagnosis of lung cancer in the subject. In one particular embodiment, the non-elevated level of serum sPLA2-IIA as compared to the baseline level correlates to a diagnosis of normal lung tissue, benign lung diseases, and/or benign solitary pulmonary nodules. In a further embodiment, the benign solitary pulmonary nodules are inflammatory pseudo tumors.

In yet another embodiment, the method for diagnosing lung cancer in the subject further includes determining a cutoff value, wherein the non-elevated level of serum sPLA2-IIA is less than the cutoff value. In one particular embodiment, the cutoff value is about 2 ng/mL.

Embodiments of diagnosing lung cancer have now been described in detail. Further embodiments directed to assessing lung cancer will now be described.

### IV. Method for Assessing Lung Cancer

In another embodiment, an in vitro method for assessing lung cancer in a patient is disclosed. The method includes: (a) contacting a portion of a sample from the patient with a specific binding agent for sPLA2-IIA, a specific binding agent for CEA, and a specific binding agent for Cyfra 21.1, (b) calculating levels of sPLA2-IIA, CEA, and Cyfra 21.1 based on the contacting in step (a), and (c) assessing as indicating lung cancer in the patient if the calculated levels of sPLA2-IIA, CEA, and/or Cyfra 21.1 are elevated relative to cutoff values of sPLA2-IIA, CEA, and Cyfra 21.1.

In one embodiment, the method includes contacting a portion of a sample from the patient with a specific binding agent for serum sPLA2-IIA, a specific binding agent for CEA, and a specific binding agent for Cyfra 21.1. Contacting a portion of the sample from the patient with such specific binding agents may be performed as set forth in the Instructions for Utilizing the Kit for Assessing Lung Cancer as described in greater detail in a later section. The sample is as previously described with regard to a biological sample. Additionally, the sample may be obtained via blood testing as previously described.

With regard to the specific binding agents for sPLA2-IIA, CEA, and Cyfra 21.1, examples of suitable specific binding agents respectively include at least one antibody having specific binding affinity to sPLA2-IIA, at least one antibody having specific binding affinity to CEA, and at least one antibody having specific binding affinity to Cyfra 21.1. Such antibody may be a capture antibody and/or a detection antibody as described in greater detail in a later section. In one particular embodiment, the sample from the patient is also contacted in step (a) with a specific binding agent for SCCA and/or with a specific binding agent for NSE. Additionally, in a further embodiment, the sample from the patient is also contacted in step (a) with a specific binding agent for ProGRP. Examples of suitable specific binding agents for SCCA, NSE, and ProGRP respectively include at least one antibody having specific binding affinity to SCCA, at least one antibody having specific binding affinity to NSE, and at least one antibody having specific binding affinity to ProGRP. Such antibody may also be a capture antibody and/or a detection antibody as described in greater detail in a later section.

The method also includes calculating levels of sPLA2-IIA, CEA, and Cyfra 21.1 based on the contacting in step (a). In one particular embodiment, levels of SCCA and NSE are also calculated in step (b). In still a further embodiment, a level of ProGRP is also calculated in step (b). Such calculations may be performed as set forth in the Instructions for Utilizing the Kit for Assessing Lung Cancer as described in greater detail in a later section.

In another embodiment, the method includes assessing as indicating lung cancer in the patient if the calculated levels of sPLA2-IIA, CEA, and/or Cyfra 21.1 are elevated relative to cutoff values of sPLA2-IIA, CEA, and Cyfra 21.1. In one particular embodiment, lung cancer is indicated if the calculated levels of at least one of sPLA2-IIA, CEA, or Cyfra 21.1 are elevated relative to their respective cutoff values. In another embodiment, lung cancer is indicated if the calculated levels of two or more of sPLA2-IIA, CEA, or Cyfra 21.1 are elevated relative to their respective cutoff values. In yet another embodiment, cancer is indicated if the calculated levels of sPLA2-IIA, CEA, and Cyfra 21.1 are elevated relative to their respective cutoff values. With regard to embodiments wherein the levels of levels of SCCA, NSE, and/or ProGRP are also calculated, lung cancer is indicated if at least one of sPLA2-IIA, CEA, Cyfra 21.1, SCCA, NSE, or ProGRP is elevated. In other embodiments, lung cancer is indicated if the calculated levels of two or more, three or more, four or more, five or more, or all of sPLA2-IIA, CEA, Cyfra 21.1, SCCA, NSE, or ProGRP are elevated. Levels of sPLA2-IIA, CEA, Cyfra 21.1, SCCA, NSE, and/or ProGRP are elevated in the sample when they are greater than the respective cutoff values of sPLA2-IIA, CEA, Cyfra 21.1, SCCA, NSE, and/or ProGRP.

In one particular embodiment, the cutoff value of sPLA2-IIA is 2.4 ng/mL, the cutoff value of CEA is 6.0 ng/mL, the cutoff value of Cyfra 21.1 is 3.3 ng/mL. With regard to embodiments wherein the levels of SCCA, NSE, and/or ProGRP are also calculated, the cutoff value of SCCA is 2.0 ng/mL, the cutoff value of NSE is 12.5 ng/mL, and the cutoff value of ProGRP is 300 pg/mL.

Embodiments of methods of assessing lung cancer have now been described in detail. Further embodiments directed to kits for assessing lung cancer will now be described.

### V. Kits for Assessing Lung Cancer

In another embodiment, a kit for assessing lung cancer is disclosed. In one particular embodiment, the kit is used to assess lung cancer in patients with solitary pulmonary nodules. Generally, the kit includes reagents for detection and/or quantification of lung cancer protein biomarkers. In one embodiment, the kit includes reagents for detection and/or quantification of sPLA2-IIA in plasma, reagents for detection and/or quantification of CEA in plasma, and reagents for detection and/or quantification of Cyfra 21.1 in plasma. In one embodiment, the kit for assessing lung cancer is an ELISA kit which may be used to detect and/or quantify sPLA2-IIA, CEA, Cyfra 21.1, SCCA, NSE, and/or ProGRP.

A. Reagents for Detection and/or Quantification of sPLA2-IIA

In one embodiment, reagents for detection and/or quantification of sPLA2-IIA include at least one antibody having specific binding affinity to sPLA2-IIA in the plasma. The antibody having specific binding affinity to sPLA2-IIA may be a capture antibody and/or a detection antibody. Each of the capture antibody and the detection antibody may have identical or different amino acid sequences. In one embodiment, the antibody is a capture antibody having specific binding affinity to sPLA2-IIA. In one particular embodiment, the capture antibody is anti-sPLA2-IIA. The capture antibody may be immobilized on a surface of a suitable substrate. For example, in one embodiment, the capture antibody is deposited directly on a surface of the substrate such that the capture antibody directly contacts at least a portion of the surface. The substrate may be a microtitre plate, a microplate, a microwell plate, and/or a strip plate. For example, in one particular embodiment, the capture antibody is immobilized on a surface of a well of the microtitre plate, microplate, microwell plate, and/or strip plate.

In another embodiment, the antibody having specific binding affinity to sPLA2-IIA is a detection antibody. In one particular embodiment, the detection antibody is free in solution. Stated another way, in one embodiment, the detection antibody is not immobilized on a surface of a suitable substrate. In one embodiment, the reagents for detection and/or quantification of sPLA2-IIA include an enzyme conjugated to the detection antibody having specific binding affinity to sPLA2-IIA in the plasma. In one particular embodiment, the detection antibody having specific binding affinity to sPLA2-IIA in the plasma is sPLA2-IIA fragment antigen. In another particular embodiment, the enzyme conjugated to the detection antibody is acetylcholinesterase. In a further embodiment, the enzyme conjugated to the detection antibody having specific binding affinity to the sPLA2-IIA in the plasma is sPLA2-IIA acetylcholinesterase-fragment antigen-binding conjugate, or sPLA2 (human Type IIA) AChE-FAb′ conjugate.

In another embodiment, the reagents for detection and/or quantification of sPLA2-IIA include a substrate of the enzyme, wherein action of the enzyme upon the substrate produces at least one detectable product. In one particular embodiment wherein the enzyme is acetylcholinesterase, the substrate is 5,5′-dithiobis-(2-nitrobenzoic acid) (i.e., Ellman's Reagent or DTNB). DTNB produces at least one detectable product upon reaction with acetylcholinesterase. More particularly, action of acetylcholinesterase upon DTNB produces NTB²⁻ dianion in water at neutral and alkaline pH. The NTB²⁻ dianion has a yellow color. The NTB²⁻ dianion is detectable and/or quantifiable in a spectrophotometer. More particularly, the NTB²⁻ dianion may be detected and/or quantified in a spectrophotometer by measuring the absorbance of visible light at 412 nm, using an extinction coeffection of 14,150 M⁻¹ cm⁻¹ for dilute solutions, and an extinction coefficient of 13,700 M⁻¹ cm⁻¹ for high salt concentrations.

The reagents for detection and/or quantification of sPLA2-IIA in plasma may also include: a polysorbate surfactant, a phosphate buffer, a wash buffer, a blocking protein, protein standard, and combinations thereof. In one embodiment, an example of a suitable polysorbate surfactant is PEG(20)sorbitan monolaurate. In one embodiment, an example of a phosphate buffer includes phosphate solution containing bovine serum albumin (i.e., BSA), sodium chloride, ethylenediaminetetraacetic acid (i.e., EDTA), and sodium azide. In one embodiment, an example of a suitable wash buffer is phosphate solution. In one embodiment, an example of a suitable blocking protein is non-specific mouse serum. Blocking proteins may be effective to reduce nonspecific binding of proteins to reaction surfaces, maximizing signal-to-noise ratio. In one embodiment, an example of a suitable protein standard is sPLA2-IIA.

B. Reagents for Detection and/or Quantification of CEA

In one embodiment, reagents for detection and/or quantification of CEA include at least one antibody having specific binding affinity to the carcinoembryonic antigen in the plasma. The antibody having specific binding affinity to CEA may be a capture antibody and/or a detection antibody. Each of the capture antibody and the detection antibody may have identical or different amino acid sequences. In one embodiment, the antibody is a capture antibody having specific binding affinity to CEA. In one particular embodiment, the capture antibody is monoclonal anti-CEA. The capture antibody may be immobilized on a surface of a suitable substrate as previously described.

In another embodiment, the antibody having specific binding affinity to CEA is a detection antibody. In one particular embodiment, the detection antibody is free in solution as previously described. The reagents for detection and/or quantification of CEA may include an enzyme conjugated to the detection antibody having specific binding affinity to CEA in the plasma. In one particular embodiment, the detection antibody having specific binding affinity to CEA in the plasma is monoclonal anti-CEA. In another particular embodiment, the enzyme conjugated to the detection antibody is horseradish peroxidase. In a further embodiment, the enzyme conjugated to the antibody having specific binding affinity to CEA in the plasma is monoclonal anti-CEA conjugated to horseradish peroxidase.

In another embodiment, the reagents for detection and/or quantification of CEA include a substrate of the enzyme, wherein action of the enzyme upon the substrate produces at least one detectable product. In one particular embodiment wherein the enzyme is horseradish peroxidase, the substrate is 3,3′,5,5′-Tetramethylbenzidine (i.e., TMB). TMB produces at least one detectable product upon reaction with horseradish peroxidase. More particularly, action of horseradish peroxidase upon TMB produces a diimine from reduction of hydrogen peroxide to water. The resulting diimine causes solution to take on a blue color. The diimine is detectable and/or quantifiable in a spectrophotometer. More particularly, the diimine may be detected and/or quantified in a spectrophotometer by measuring the absorbance of visible light at 450 nm.

The reagents for detection and/or quantification of CEA may also include a stopping solution. Stopping solutions may be effective to termination action of the enzyme upon the substrate. In one particular embodiment wherein the enzyme is horseradish peroxidase and the substrate is TMB, an example of a suitable stopping solution is an acid. More particularly, in another particular embodiment, an example of a suitable stopping solution is HCl. The reagents for detection and/or quantification of CEA may also include a protein standard. In one particular embodiment, an example of a suitable protein standard is CEA standard.

C. Reagents for Detection and/or Quantification of Cyfra 21.1

In one embodiment, reagents for detection and/or quantification of Cyfra 21.1 include at least one antibody having specific binding affinity to Cyfra 21.1 in the plasma. The antibody having specific binding affinity to Cyfra 21.1 may be a capture antibody and/or a detection antibody. Each of the capture antibody and the detection antibody may have identical or different amino acid sequences. In one embodiment, the antibody is a capture antibody having specific binding affinity to Cyfra 21.1. In one particular embodiment, the capture antibody is anti-Cyfra 21.1 antibody. The capture antibody may be immobilized on a surface of a suitable substrate as previously described.

In another embodiment, the antibody having specific binding affinity to Cyfra 21.1 is a detection antibody. In one particular embodiment, the detection antibody is free in solution as previously described. The reagents for detection and/or quantification of Cyfra 21.1 may include an enzyme conjugated to the detection antibody having specific binding affinity to Cyfra 21.1 in the plasma. In one particular embodiment, the detection antibody having specific binding affinity to Cyfra 21.1 in the plasma is monoclonal anti-Cyfra 21.1 antibody. In another particular embodiment, the enzyme conjugated to the detection antibody is horseradish peroxidase. In a further embodiment, the enzyme conjugated to the antibody having specific binding affinity to Cyfra 21.1 in the plasma is horseradish peroxidase conjugated to anti-Cyfra 21.1 antibody.

In another embodiment, the reagents for detection and/or quantification of Cyfra 21.1 include a substrate of the enzyme, wherein action of the enzyme upon the substrate produces at least one detectable product. In one particular embodiment wherein the enzyme is horseradish peroxidase, the substrate is TMB. Action of horseradish peroxidase upon TMB and detection and/or quantification of TMB is as previously described.

The reagents for detection and/or quantification of Cyfra 21.1 may also include: a stopping solution, a protein standard, a wash buffer, at least one diluent, and combinations thereof.

In one embodiment, an example of a suitable stopping solution is an acid. An example of a suitable stopping solution for use with TMB and horseradish peroxidase is HCl. In one embodiment, an example of a suitable standard is Cyfra 21.1 standard.

D. Reagents for Detection and/or Quantification of SCCA

In one embodiment, the kit for assessing lung cancer further includes reagents for detection and/or quantification of SCCA in plasma. In one embodiment, reagents for detection and/or quantification of SCCA include at least one antibody having specific binding affinity to SCCA in the plasma. The antibody having specific binding affinity to SCCA may be a capture antibody and/or a detection antibody. Each of the capture antibody and the detection antibody may have identical or different amino acid sequences. In one embodiment, the antibody is a capture antibody having specific binding affinity to SCCA. In one particular embodiment, the capture antibody is anti-human SCCA 1. The capture antibody may be immobilized on a surface of a suitable substrate as previously described.

In another embodiment, the antibody having specific binding affinity to SCCA is a detection antibody. In one particular embodiment, the detection antibody is free in solution as previously described. In one embodiment, the detection antibody is anti-human SCCA 1. The reagents for detection and/or quantification of SCCA may include an enzyme complex capable of conjugating to the detection antibody having specific binding affinity to SCCA in the plasma. In one particular embodiment, the enzyme is peroxidase. In a further embodiment, the enzyme complex capable of conjugating to the detection antibody having specific binding affinity to SCCA in the plasma is an avidin-biotin-peroxidase complex.

In another embodiment, the reagents for detection and/or quantification of SCCA include a substrate of the enzyme, wherein action of the enzyme upon the substrate produces at least one detectable product. In one particular embodiment wherein the enzyme is peroxidase, the substrate is TMB. Action of horseradish peroxidase upon TMB and detection and/or quantification of TMB is as previously described.

The reagents for detection and/or quantification of SCCA may also include: a stopping solution, a protein standard, at least one diluent, and combinations thereof. In one embodiment, an example of a suitable stopping solution is an acid. An example of a suitable stopping solution for use with TMB and peroxidase is HCl. In one embodiment, an example of a suitable standard is SCCA standard.

E. Reagents for Detection and/or Quantification of NSE

In one embodiment, the kit for assessing lung cancer further includes reagents for detection and/or quantification of NSE in plasma. In one embodiment, reagents for detection and/or quantification of NSE include at least one antibody having specific binding affinity to NSE in the plasma. The antibody having specific binding affinity to NSE may be a capture antibody and/or a detection antibody. Each of the capture antibody and the detection antibody may have identical or different amino acid sequences. In one embodiment, the antibody is a capture antibody having specific binding affinity to NSE. In one particular embodiment, the capture antibody is NSE antibody. The capture antibody may be immobilized on a surface of a suitable substrate as previously described. For example, in one particular embodiment, the substrate may contain a streptavidin coating. In this particular embodiment, the capture antibody may be biotinylated. For example, in one particular embodiment, the substrate is a streptavidin-coated microplate and the capture antibody is a biotinylated NSE antibody.

In another embodiment, the antibody having specific binding affinity to NSE is a detection antibody. In one particular embodiment, the detection antibody is free in solution as previously described. In another particular embodiment, the detection antibody is anti-human NSE specific monoclonal antibody. The reagents for detection and/or quantification of NSE may include an enzyme conjugated to the detection antibody having specific binding affinity to NSE in the plasma. In one particular embodiment, the enzyme conjugated to the detection antibody is horseradish peroxidase. In a further embodiment, the enzyme conjugated to the antibody having specific binding affinity to NSE in the plasma is horseradish peroxidase labeled anti-human NSE specific monoclonal antibody.

The reagents for detection and/or quantification of NSE may include a substrate of the enzyme, wherein action of the enzyme upon the substrate produces at least one detectable product. In one particular embodiment wherein the enzyme is horseradish peroxidase, the substrate is TMB. Action of horseradish peroxidase upon TMB and detection and/or quantification of TMB is as previously described.

The reagents for detection and/or quantification of NSE may also include: a stopping solution, a protein standard, a wash buffer, and combinations thereof. In one embodiment, an example of a suitable stopping solution is an acid. An example of a suitable stopping solution for use with TMB and horseradish peroxidase is H₂SO₄. In one embodiment, an example of a suitable standard is NSE standard. In one embodiment, an example of a suitable wash buffer is a phosphate buffered saline solution containing a surfactant.

F. Reagents for Detection and/or Quantification of ProGRP

In one embodiment, the kit for assessing lung cancer further includes reagents for detection and/or quantification of ProGRP in plasma. In one embodiment, reagents for detection and/or quantification of ProGRP in plasma include at least one antibody having specific binding affinity to ProGRP in the plasma. The antibody having specific binding affinity to ProGRP may be a capture antibody and/or a detection antibody. Each of the capture antibody and the detection antibody may have identical or different amino acid sequences. In one embodiment, the antibody is a capture antibody having specific binding affinity to ProGRP. In one particular embodiment, the capture antibody is human ProGRP monoclonal antibody. The capture antibody may be immobilized on a surface of a suitable substrate as previously described.

In another embodiment, the antibody having specific binding affinity to ProGRP is a detection antibody. In one particular embodiment, the detection antibody is free in solution as previously described. In one embodiment, the detection antibody is human ProGRP monoclonal antibody. The detection antibody may be labeled. For example, in one embodiment, the detection antibody is human ProGRP labeled with biotin. The reagents for detection and/or quantification of ProGRP may include a labeled enzyme capable of conjugating to the labeled detection antibody having specific binding affinity to ProGRP in the plasma. In one particular embodiment wherein the detection antibody is human ProGRP labeled with biotin, the enzyme is horseradish peroxidase labeled with streptavidin. Action of horseradish peroxidase upon TMB and detection and/or quantification of TMB is as previously described.

The reagents for detection and/or quantification of ProGRP may include a substrate of the enzyme, wherein action of the enzyme upon the substrate produces at least one detectable product. In one particular embodiment wherein the enzyme is horseradish peroxidase, the substrate is TMB. Action of horseradish peroxidase upon TMB and detection and/or quantification of TMB is as previously described.

The reagents for detection and/or quantification of ProGRP may also include: a stopping solution, a protein standard, and combinations thereof. In one embodiment, an example of a suitable stopping solution is an acid. In one embodiment, an example of a suitable standard is ProGRP standard.

In one particular embodiment, a kit for assessing lung cancer in patients having solitary pulmonary nodules is disclosed. The kit includes reagents for detection and/or quantification of sPLA2-IIA, wherein the reagents for detection and/or quantification of sPLA2-IIA in plasma include a capture antibody having specific binding affinity to the sPLA2-IIA in the plasma, an enzyme conjugated to a detection antibody having specific binding affinity to the sPLA2-IIA in the plasma, and a substrate of the enzyme conjugated to the detection antibody having specific binding affinity to the sPLA2-IIA in the plasma, wherein action of the enzyme conjugated to the antibody having specific binding affinity to the sPLA2-IIA in the plasma upon the substrate produces at least one detectable product. The kit also includes reagents for detection and/or quantification of CEA in plasma, wherein the reagents for detection and/or quantification of CEA in plasma include a capture antibody having specific binding affinity to the CEA in the plasma, an enzyme conjugated to a detection antibody having specific binding affinity to the CEA in the plasma, and a substrate of the enzyme conjugated to the detection antibody having specific binding affinity to the CEA in the plasma, wherein action of the enzyme conjugated to the detection antibody having specific binding affinity to the CEA in the plasma upon the substrate produces at least one detectable product. The kit also includes reagents for detection and/or quantification of Cyfra 21.1 in plasma, wherein the reagents for detection and/or quantification of Cyfra 21.1 in plasma includes a capture antibody having specific binding affinity to the Cyfra 21.1 in the plasma, an enzyme conjugated to a detection antibody having specific binding affinity to the Cyfra 21.1 in the plasma, and a substrate of the enzyme conjugated to the detection antibody having specific binding affinity to the Cyfra 21.1 in the plasma, wherein action of the enzyme conjugated to the detection antibody having specific binding affinity to the Cyfra 21.1 in the plasma upon the substrate produces at least one detectable product.

Each of the reagents described herein may be applied to detect and/or quantify any of the lung cancer protein biomarkers described herein where suitable. For example, with regard to Cyfra 21.1, the reagents for detection and/or quantification of Cyfra 21.1 may include at least one diluent. Where suitable, such diluent may also be included in the reagents for detection and/or quantification of sPLA2-IIA, SCCA, NSE, and/or ProGRP.

### G. Instructions for Utilizing the Kit for Assessing Lung Cancer

The kit may also include instructions to provide guidance on utilizing the kit for assessing lung cancer. More particularly, the kit may include instructions to provide guidance on detecting and/or quantifying lung cancer protein biomarkers, e.g., sPLA2-IIA, CEA, Cyfra 21.1, SCCA, NSE, and/or ProGRP. Such instructions may generally include information related to performing ELISA and/or calculating results.

Generally, such instructions for performing ELISA may include the following: (1) securing a microplate in a microplate holder; (2) dispensing standards, controls, and/or samples into wells of the microplate; (3) dispensing the enzyme conjugated to a detection antibody having specific binding affinity to sPLA2-IIA, CEA, Cyfra 21.1, SCCA, NSE, and/or ProGRP into each well; (4) Mixing thoroughly; (5) incubating at room temperature; (6); removing contents from the wells; (7) optionally rinsing the wells with wash buffer; (8) adding the substrate to each well; (9) incubating at room temperature; (10) optionally terminating the enzymatic reaction by adding stopping solution to each well; and (11) reading the optical density with a microplate reader within 10 minutes of adding the stopping solution. The kit may also include instructions for calculating results. Such instructions may include the following: (1) calculating average absorbance values for each set of standards, controls, and patient samples; (2) constructing a standard curve by plotting the mean absorbance obtained from each standard against its concentration with absorbance value on the vertical (Y) axis and concentration on the horizontal (X) axis; and (3) using the mean absorbance value for each sample to determine the corresponding concentration from the standard curve. Alternatively, the results may be calculated automatically using a 4 Parameter Logistics Curve Fit.

### H. Utilizing the Kit for Assessing Lung Cancer

An in vitro method for assessing lung cancer in a patient including utilizing the kit previously described is disclosed. The method includes (a) contacting a portion of a sample from the patient with the reagents for detection and/or quantification of sPLA2-IIA in plasma, the reagents for detection and/or quantification of CEA in plasma, and the reagents for detection and/or quantification of Cyfra 21.1 in plasma. The reagents for detection and/or quantification of sPLA2-IIA, CEA, and Cyfra 21.1 are as previously described. The method also includes (b) calculating levels of sPLA2-IIA, CEA, and Cyfra 21.1 fragment based on the contacting in step (a). Additionally, the method includes (c) assessing as indicating lung cancer in the patient if the calculated levels of sPLA2-IIA, CEA, and Cyfra 21.1 are elevated relative to cutoff values of sPLA2-IIA, CEA, and Cyfra 21.1.

In one embodiment, step (a) further includes contacting a portion of the sample from the patient with reagents for detection and/or quantification of SCCA and reagents for detection and/or quantification of specific binding agent for NEA. Step (a) may also further include contacting a portion of the sample from the patient with reagents for detection and/or quantification of ProGRP. The reagents for detection and/or quantification of SCCA, NEA, and ProGRP are as previously described.

Embodiments of kits for assessing lung cancer have now been described in detail. Further embodiments directed to kits for assessing prostate cancer will now be described.

### VI. Kits for Assessing Prostate Cancer

In one embodiment, a kit for assessing prostate cancer is disclosed. The kit includes reagents for detection and/or quantification of serum secretory phospholipase A₂-IIA in plasma and reagents for detection and/or quantification of PSA. In one particular embodiment, the reagents are utilized to detect and/or quantify PSA in serum.

Embodiments of kits for assessing prostate cancer have now been described in detail. Further embodiments directed to methods for assessing prostate cancer will now be described.

### VII. Methods for Assessing Prostate Cancer

In another embodiment, an in vitro method for assessing prostate cancer in a patient is disclosed. The method includes: (a) contacting a portion of a sample from the patient with a specific binding agent for sPLA2-IIA and a specific binding agent for PSA, (b) calculating levels of sPLA2-IIA and PSA based on the contacting in step (a), and (c) assessing as indicating prostate cancer in the patient if the calculated levels of sPLA2-IIA and PSA are elevated relative to cutoff values of sPLA2-IIA and PSA.

### VIII. Managing Treatment of Lung Cancer

In another embodiment, a method of managing treatment of a patient suspected of having lung cancer is disclosed. In one embodiment, the method includes (a) assessing lung cancer in vitro in the patient by calculating in a plasma sample from the patient a level of sPLA2-IIA, and optionally calculating in the plasma sample levels of CEA and Cyfra 21.1, wherein an elevated level of sPLA2-IIA, CEA, and/or Cyfra 21.1 relative to cutoff values of sPLA2-IIA, CEA, and Cyfra 21.1 are indicative of lung cancer. The method may also include (b) treating the patient for lung cancer where lung cancer is indicated, wherein the calculated level of sPLA2-IIA, CEA, and/or Cyfra 21.1 in step (a) is a baseline level(s). Additionally, the method may also include (c) determining treatment response of the patient by calculating in a sample from the patient a post-treatment level of sPLA2-IIA, CEA, and/or Cyfra 21.1, wherein a decreased post-treatment level compared to the baseline level is indicative of a successful treatment response. In one embodiment, the treatment includes surgical resection, chemotherapy, radiation therapy, biological therapy, and combinations thereof.

In another embodiment, a method of managing treatment in a patient suffering from lung cancer is disclosed. The method includes (a) establishing a baseline level of sPLA2-IIA in a plasma sample from the patient and optionally one or more additional markers of lung cancer; (b) initiating treatment; and (c) serially monitoring the level of sPLA2-IIA in the patient during treatment, wherein the treatment is managed to decrease the level of sPLA2-IIA in the patient.

Without being bound by the theory, it is believed that patients with elevated levels of sPLA2-IIA in plasma relative to a cutoff value of 2.4 ng/mL may be good candidates for therapies targeting HER/HER2-PI3K-Akt-NF-κB signaling pathways, e.g., Erlotinib targeting EGFR, Lapatinib targeting EGFR and HER2, and Bortezomib targeting NF-κB. Additionally, it is believed that blood tests involving detection of sPLA2-IIA in plasma via a blood test will provide a tool for personalized therapy against lung cancer.

### EXAMPLES

The following non-limiting examples illustrate the methods of the present disclosure.

### Example 1

### Regulation of Human sPLA2-IIA Gene Expression Mediated by the EGFR/HER2-Elicited Pathways

Experimental Protocol.

The role of sPLA2-IIA gene regulation in prostate cancer cells via the HER/HER2-PI3K-Akt-NF-κB pathway was investigated. A reporter assay was performed by transiently transfecting sPLA2-IIA(-800)-Luc (˜0.25 μg/well) reporter in LNCaP-AI cells (˜10⁵ cells/well in 12-well plate). The cells were then treated with epidermal growth factor (hereinafter “EGF”) (˜100 ng/mL) without or with EGFR inhibitors Erlotinib (˜20 μM) and Gefitinib (˜20 μM), EGFR/HER2 dual inhibitors Lapatinib (˜20 μM) and CI-1033 (˜8 μM), phosphoinositide 3-kinase (hereinafter “PI3K”) inhibitor LY294002 (˜20 μM), and NF-κB inhibitor Bortezomib (˜20 μM) for about 24 hours. A luciferase assay was performed according to a standard protocol with Renilla luciferase as an internal control. Another reporter assay was performed wherein LNCaP-AI cells were starved in 1% stripped medium for about 24 hours. The cells were the treated with Erlotinib (˜20 μM), Gefitinib (˜20 μM), Lapatinib (˜20 μM), CI-1033 (˜8 μM), LY294002 (˜20 μM), Bortezomib (˜20 μM), and/or Heregulin-α (˜50 ng/mL) without or with EGF (˜100 ng/mL) for about 24 hours.

Cell extracts were then prepared and subjected to western blot analysis for sPLA2-IIA, P-Akt, Akt, and β-actin. Briefly, aliquots of samples with the same amount of protein, determined using the Bradford assay (BioRad, Hercules, Calif.), were mixed with loading buffer (final concentrations of 62.5 mM Tris-HCl, pH 6.8, 2.3% sodium dodecyl sulfate, 100 mM dithiothreitol, and 0.005% bromophenol blue), boiled, fractionated in a sodium dodecyl sulfate-polyacrylamide gel electrophoresis, and transferred onto a 0.45-μm nitrocellulose membrane (BioRad). The filters were blocked with 2% fat-free milk in PBS, and probed with primary antibody in phosphate buffered saline containing 0.1% Tween 20 (i.e., PBST) and 1% fat-free milk. The membranes were then washed four times in PBST and incubated with horseradish peroxidase-conjugated secondary antibody (BioRad) in PBST containing 1% fat-free milk. After washing four times in PBST, the membranes were visualized using the ECL Western Blotting Detection System (Amersham Co, Arlington Height, Ill.).

Finally, LNCaP-AI cells were starved in 1% stripped medium for about 24 hours. The cells were then treated with Erlotinib (˜20 μM), Gefitinib (˜20 μM), Lapatinib (˜20 μM), CI-1033 (˜8 μM), LY294002 (˜20 μM), and Bortezomib (˜20 μM) for about 24 hours. Cell culture medium was collected from each sample and subjected to ELISA for sPLA2-IIA. The condition medium samples were diluted 10 times for ELISA. The average of duplicate samples was converted to nanogram per milliliter against standard curve. The data represent one of five repeated experiments.

Experimental Results.

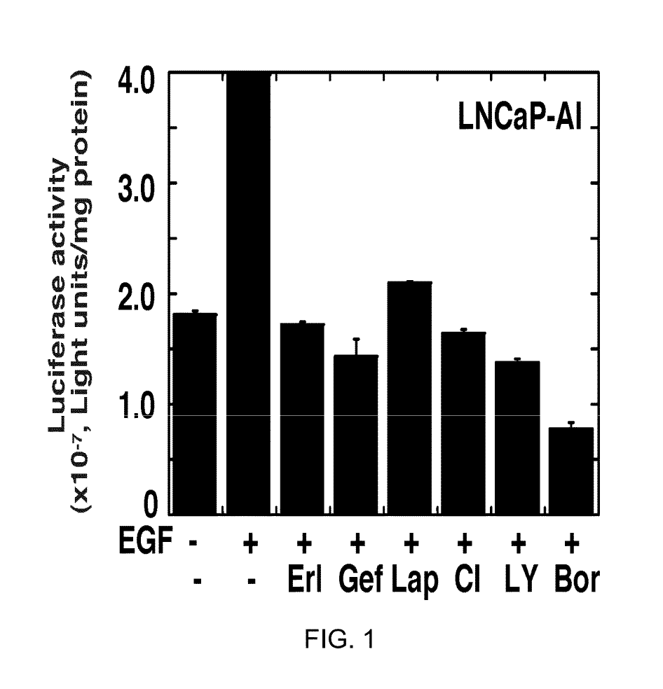

As shown in FIG. 1, EGF significantly stimulated the promoter activity of sPLA2-IIA gene, which was blocked by EGFR inhibitors Erlotinib and Gefitinib, EGFR/HER2 dual inhibitors Lapatinib and CI-1033, PI3K inhibitor LY294002, and NF-κB inhibitor Bortezomib. This data indicates that the elevated signaling of the HER/HER2-PI3K-Akt-NF-κB pathway upregulates expression of sPLA2-IIA gene at the transcriptional level. Data are presented as the mean (±SD) of duplicative values of a representative experiment that was independently repeated for five times.

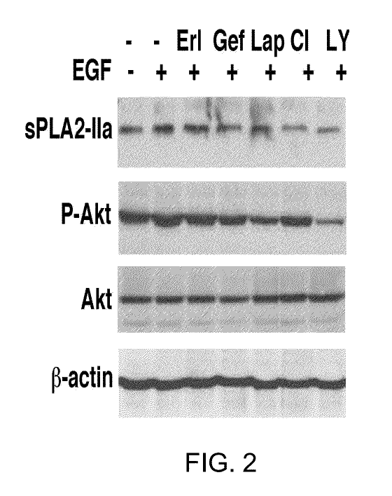

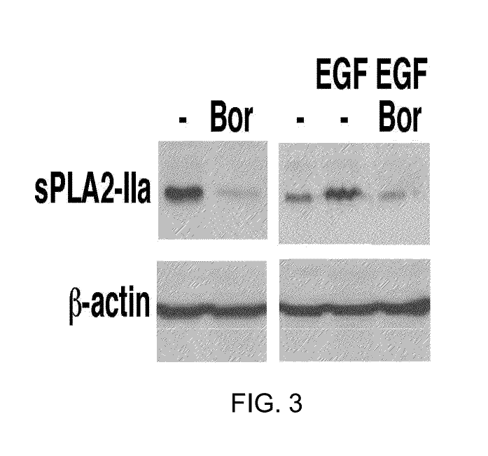

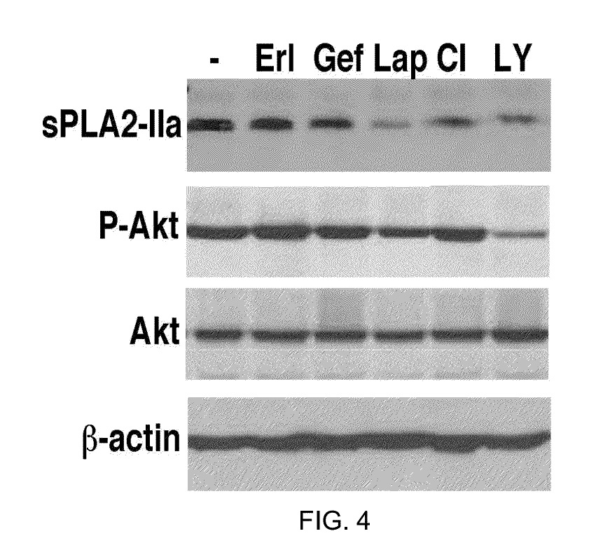

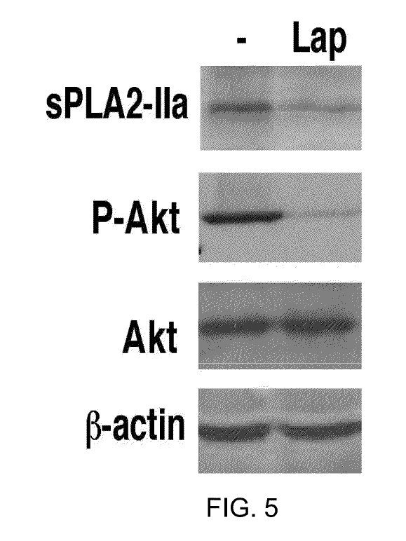

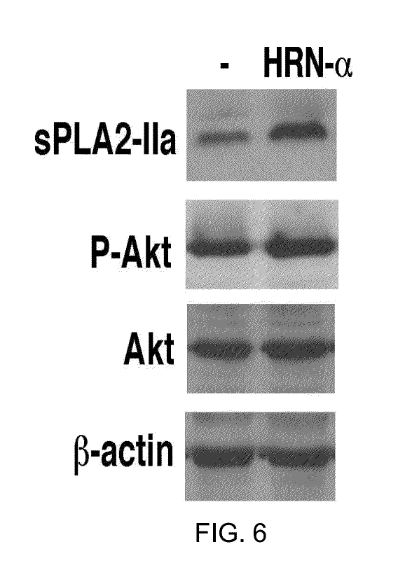

As shown in FIGS. 2 and 3, EGF stimulated sPLA2-IIA expression. Moreover, as shown in FIGS. 2-4, among the inhibitors examined, Lapatinib, LY294002 and Bortezomib dramatically downregulated sPLA2-IIA protein expression in both basal states and in the setting of EGF-induced expression, whereas Erlotinib, Gefitinib, and CI-1033 had a moderate impact on sPLA2-IIA protein expression. As shown in FIG. 5, sPLA2-IIA was also expressed in LAPC-4 and DU145 cells, but not PC-3 cells, which were inhibited by Lapatinib via blocking PI3K-Akt signaling. Finally, as shown in FIG. 6, HER3 ligand Heregulin-α enhanced Akt phosphorylation and sPLA2-IIA expression via PI3K-Akt signaling in LNCaP cells.

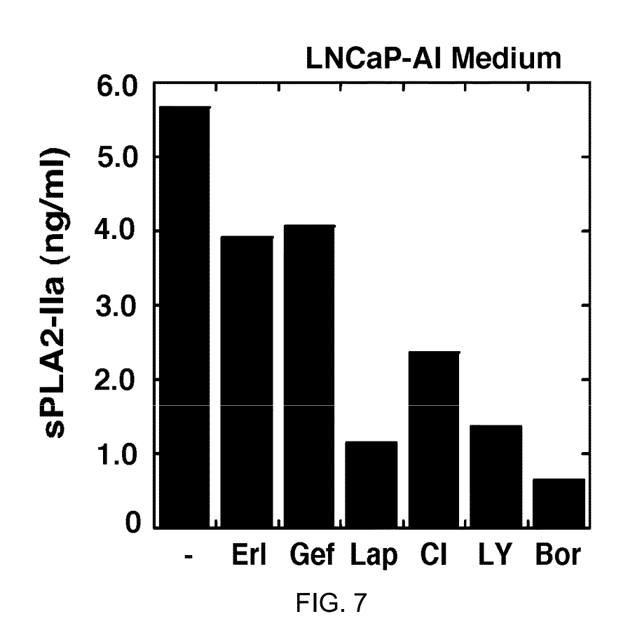

As shown in FIG. 7, Lapatinib, LY294002, and Bortezomib significantly inhibited sPLA2-IIA secretion, whereas Erlotinib, Gefitinib and CI-1033 had a moderate effect in LNCaP-AI cells.

### Example 2

### SPLA2-IIA Gene is Overexpressed in Androgen-Independent LNCaP-AI Cells

Experimental Protocol.

Expression levels of sixteen thousand genes in LNCaP-AI cells, an androgen-independent cell line, and LNCaP cells, an androgen-dependent cell line, were compared using DNA oligonucleotide microarray analysis. The androgen-independent cell line was developed from its parental androgen-dependent cell line.

The expression levels of sPLA2-IIA in LNCaP-AI cells and LNCaP cells were determined by real-time RT-PCR analysis at the mRNA level and by western blot analysis at the protein level. Additionally, quantitative analyses of the level of sPLA2-IIA in LNCaP-AI cells and LNCaP cells was performed by ELISA assay. More specifically, the LNCaP-AI cells and LNCaP cells (500,000 cells/well in 6 well plate) were cultured in stripped medium for about 2 days. The medium samples were then collected and subjected to ELISA analysis using human sPLA2 type IIa EIA kit, Catalog No. 585000 (Cayman Chemical Company, Ann Arbor, Mich.).

Experimental Results.

As shown in Table 1 below, sPLA2-IIA, Vav3, and p21/WAF were overexpressed in the LNCaP-AI cell line. Overexpression of these genes in the LNCaP-AI cell line implicates that elevated activities of these genes support androgen-independent growth in prostate cancer cells.

TABLE 1
Overexpression of sPLA2-IIA, Vav3, and p21/WAF in LNCaP-AI Cells

| | | | | | | | | UniGene |
|---|---|---|---|---|---|---|---|---|
| | LNCaP | Spot | Spot | AI vs. | Spot | Spot | | ID/Gene |
| Name | vs. AI | Norm | Norm | LNCaP | Norm | Norm | Comments | Symbol |
| H011554 | 8.8 | 4 | 1169 | 8.4 | 1300 | 5 | Vav3 | 267659/ |
| | | | | | | | oncogene | VAV3 |
| H002863 | 5.5 | 348 | 10935 | 4.5 | 12971 | 282 | sPLA2-IIA | 76422/ |
| | | | | | | | | PLA2G2A |
| H002239 | 4.3 | 452 | 5858 | 2.2 | 4285 | 490 | Cyclin- | 179665/ |
| | | | | | | | dependent | CDKN1A |
| | | | | | | | kinase | |
| | | | | | | | inhibitor 1A | |
| | | | | | | | (p21, Clp1 | |
| H002149 | 4.3 | 1028 | 13556 | 2.0 | 14941 | 1755 | Cyclin- | 179665/ |
| | | | | | | | dependent | CDKN1A |
| | | | | | | | kinase | |
| | | | | | | | inhibitor 1A | |
| | | | | | | | (p21, Clp1 | |

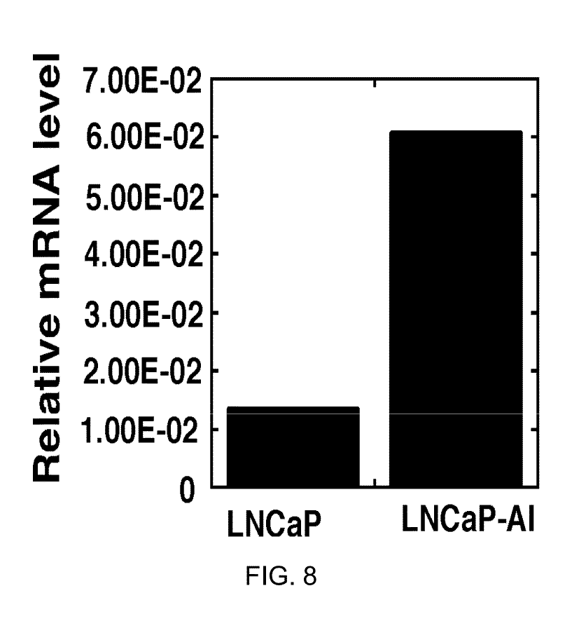

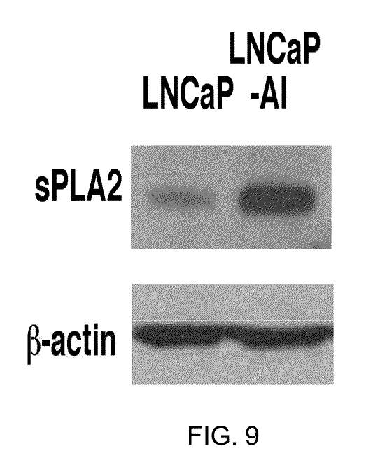

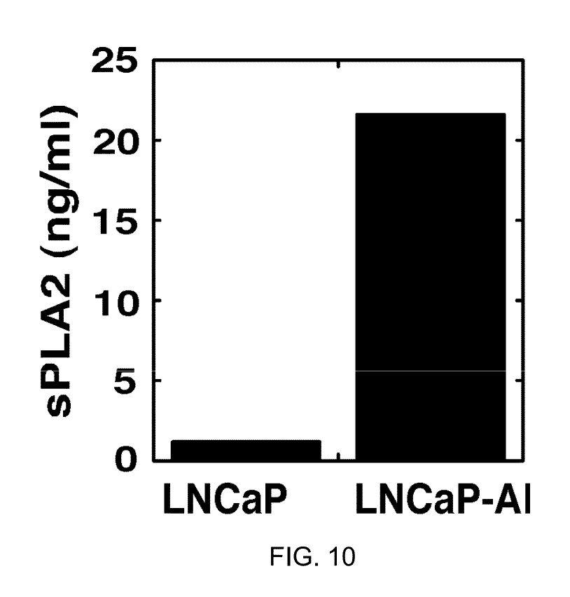

As shown in FIGS. 8 and 9, sPLA2-IIA overexpression in LNCaP-AI cells was respectively confirmed by real-time RT-PCR analysis and western blot analysis. Additionally, as shown in FIG. 10, LNCaP-AI cells secrete a greater amount of sPLA2-IIA into the medium as compared to the amount of sPLA2-IIA secreted by LNCaP into the medium.

### Example 3

### Role of sPLA2-IIA on Growth of Prostate Cancer Cells

Experimental Protocol.

The role of sPLA2-IIA in the growth of prostate cancer cells was studied by MTT assay. Specifically, LNCaP-AI cells were cultured in 10% stripped medium in the presence of EGF (ng/mL) or sPLA2-IIA (ng/mL) for about 4 days, followed by performance of an MTT assay. The role of sPLA2-IIA on prostate cancer cell growth was also studied by blocking the activity of sPLA2-IIA in LNCaP-AI cells. The activity of sPLA2-IIA was blocked in LNCaP-AI cells by administering the peptide inhibitor cFLYSYR (μM) or c(NapA)R (μM) for about 4 days, followed by performance of an MTT assay.

Experimental Results.

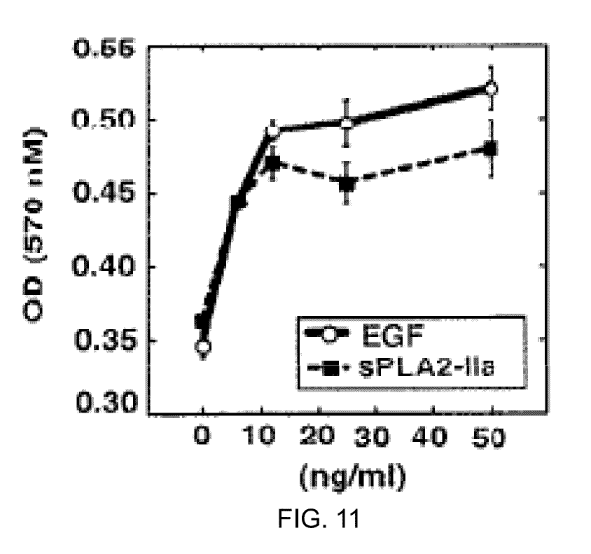

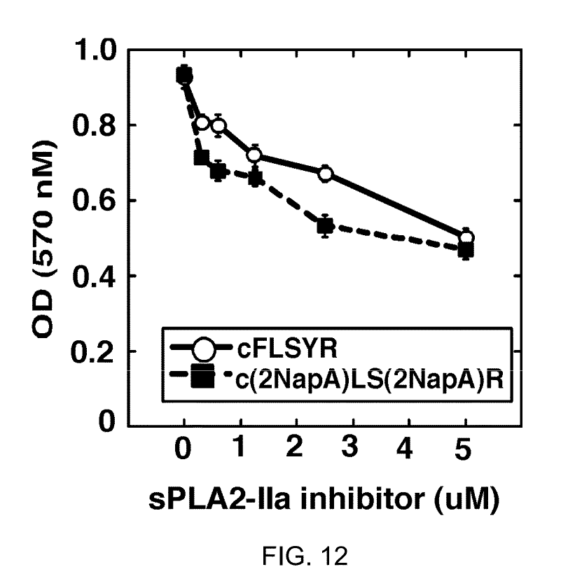

As shown in FIG. 11, sPLA2-IIA functions as a growth factor and stimulates prostate cancer cell growth in an androgen independent manner in LNCaP-AI cells. As shown in FIG. 12, blocking the activity of sPLA2-IIA in LNCaP-AI cells significantly inhibited prostate cancer cell growth. Thus, these data confirm that sPLA2-IIA plays a role in the growth of prostate cancer cells and also indicate that sPLA2-IIA overexpression contributes to prostate cancer tumorigenesis and progression.

### Example 4

### Serum sPLA2-IIA is Elevated in Prostate Cancer Patients

Experimental Protocol.

A series of studies were conducted in which serum sPLA2-IIA levels were examined in prostate cancer patients in comparison to healthy donors. In one study, (hereinafter “the 43 study”), 43 plasma samples from prostate cancer patients were obtained from the University of Cincinnati Cancer Center Tumor Bank (Cincinnati, Ohio). Of the 43 plasma samples from prostate cancer patients, 13 plasma samples had Gleason scores of about 8 to about 10 and 30 plasma samples had Gleason scores of about 6 to about 7. Additionally, 20 plasma samples from healthy donors were obtained from the Cincinnati Hoxworth Blood Center (Cincinnati, Ohio). All plasma samples were diluted ten times and then subjected to duplicate ELISA analysis using the kit previously described. The average of the duplicate sample was calculated to present as pg/mL based on the standard curve of each experiment.

Immunohistochemistry analysis of sPLA2-IIA expression in prostate cancer and benign prostatic hyperplasia (hereinafter “BPH”) specimens was also performed. BPH specimens served as a control. Prostate cancer and BPH specimens were obtained as a prostate disease spectrum tissue array (Biomatrix US, Rockville, Md.). Briefly, paraffin-embedded tissue sections were deparaffinized in xylene, rehydrated in graded alcohol, and transferred to phosphate buffered saline (hereinafter “PBS”). The slides were treated with a citric acid-based antigen-retrieval buffer (DAKO Co, Carpinteria, Calif.), followed by 3% H₂O₂ in methanol, incubated in blocking buffer (5% BSA and 5% horse serum in PBS) and then in the blocking buffer containing first antibody. After washing, the slides were incubated with a biotinylated secondary antibody (BioGenex Laboratories, San Ramon, Calif.), followed by washing and incubation with the streptavidin-conjugated peroxidase (BioGenex) and mounted with Universal Mount mounting medium (Fisher Scientific, Pittsburgh, Pa.).

Experimental Results.

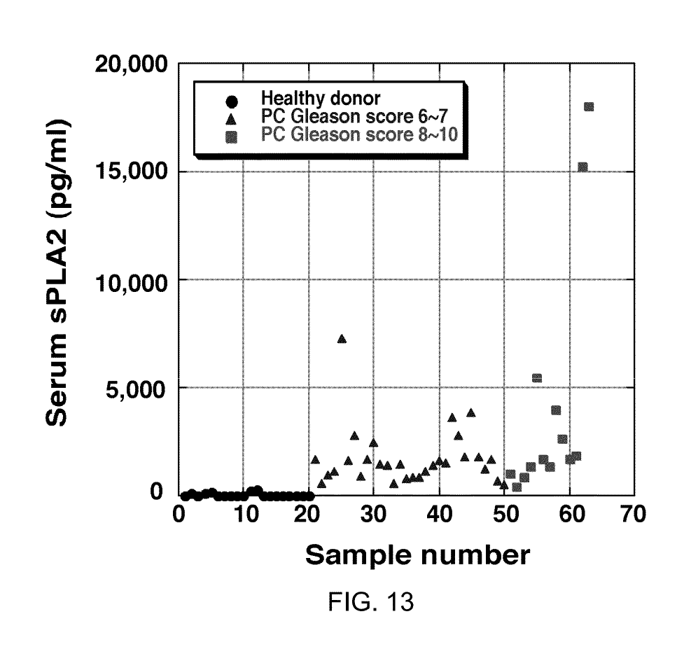

As shown in FIG. 13, in the 43 study, all prostate cancer patients showed an elevated level of serum sPLA2-IIA as compared to the healthy donors. The levels of sPLA2-IIA in prostate cancer patients ranged from ˜400 pg/mL to ˜18,000 pg/mL. Additionally, sPLA2-IIA was not detected in 15 of the plasma samples from the healthy donors and was less than ˜275 pg/mL in 5 of the plasma samples from the healthy donors. The sPLA2-IIA levels were correlated significantly with the presence of prostate cancer (p=0.0024, unpaired t test). Moreover, among the 20 healthy donors, 6 were greater than 60 years of age and 4 were greater than 50 years of age indicating that age was not a contributing factor for higher sPLA2-IIA levels in samples from cancer patients.

Also shown in FIG. 13, further analysis revealed that sPLA2-IIA levels were significantly higher in samples from prostate cancers with high Gleason score (˜8 to ˜10) than those with intermediate Gleason score (˜6 to ˜7) (p=0.0252, unpaired t test). Moreover, as shown in Table 2 below, sPLA2-IIA levels were correlated with the pathological stages of prostate cancer. Specifically, the levels in samples from patients with T3 cancer were significantly higher than those with T2 cancer (p=0.0298, nonparametric test). These results were confirmed by association analysis using the optimum cutoff value of serum sPLA2-IIA (˜1.0 ng/mL) determined by ROC curve analysis. The two samples with the highest serum sPLA2 levels (˜15,209 pg/mL and ˜18,003 pg/mL) originated from patients with stage T3 disease in prostate cancers with high Gleason scores of ˜9 and ˜10.

TABLE 2
Correlation of sPLA2-IIA Levels with Stages of Prostate Cancer

| PC stage | <1000 pg/ml | >1000 pg/ml | Total case # |
|---|---|---|---|
| T2 | 6 (35%) | 11 (65%) | 17 |
| T3 | 0 | 16 (100%) | 16 |

Fisher's exact test: P = 0.01

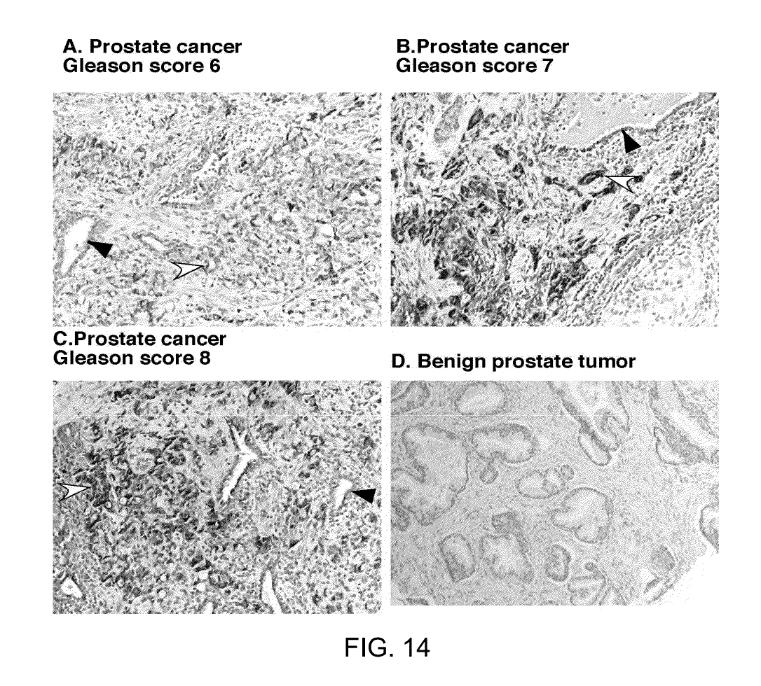

As shown in FIG. 14, immunohistochemistry staining also demonstrated elevated expression levels of sPLA2-IIA in tumor specimens with high Gleason scores and advanced cancer stage. Moreover, none of BPH specimens examined were significantly positive for sPLA2-IIA staining. As shown in (A) of FIG. 14, moderate cytoplasmic granular staining was demonstrated in a lesion of Gleason score of ˜6. As shown in (B) of FIG. 14, a strong and diffuse cytoplasmic granular staining was demonstrated in a lesion of Gleason score of ˜7. Additionally, as shown in (C) of FIG. 14, the strongest staining was demonstrated in a lesion of Gleason score of ˜8. Moreover, as shown in (D) of FIG. 14, BPH specimens were negative for sPLA2-IIA staining.

### Example 5

### Serum sPLA2-IIA is Elevated in Lung Cancer Patients

Experimental Protocol.

Serum sPLA2-IIA levels were examined in lung cancer patients in comparison with healthy donors. Specifically, 10 plasma samples from lung cancer patients were obtained from the University of Cincinnati Cancer Center Tumor Bank. 20 plasma samples from healthy donors were obtained from the Cincinnati Hoxworth Blood Center. All plasma samples were diluted ten times and then subjected to duplicate ELISA analysis using the kit previously described. The average of the duplicate sample was calculated to present as pg/mL based on the standard curve of each experiment.

Serum sPLA2-IIA levels were also examined in lung cancer patients in comparison with patients with benign nodules and plasma samples were obtained from the University of Cincinnati Cancer Center Tumor Bank. Additionally, serum sPLA2-IIA levels were also examined in lung cancer patients in comparison with healthy donors and heavy smokers without lung cancer and plasma samples were obtained from the “Genetic Epidemiology of Lung Cancer” Project, a family lung cohort study. Plasma samples from healthy donors were obtained form the Cincinnati Hoxworth Blood Center. All plasma samples were diluted ten times and then subjected to duplicate ELISA analysis using the kit previously described. The concentration of sPLA2-IIA in plasma was tested in duplicate and determined against a standard curve for each ELISA assay.

Experimental Results.

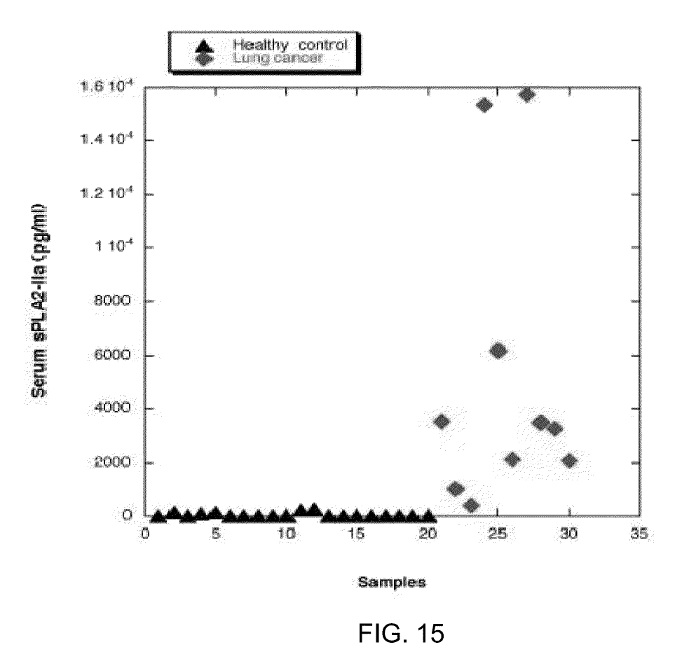

As shown in FIG. 15, lung cancer patients showed significantly elevated levels of serum sPLA2-IIA as compared to the healthy donors (P<0.001) analyzed by one-way Analysis of Variance (hereinafter “ANOVA”). Additionally, sPLA2-IIA was not detected in 15 of the plasma samples from the healthy donors and was less than ˜275 pg/mL in 5 of the plasma samples from the healthy donors. Moreover, among the 20 healthy donors, 6 were greater than 60 years of age and 4 were greater than 50 years of age indicating that age was not a contributing factor for higher sPLA2-IIA levels in samples from lung cancer patients.

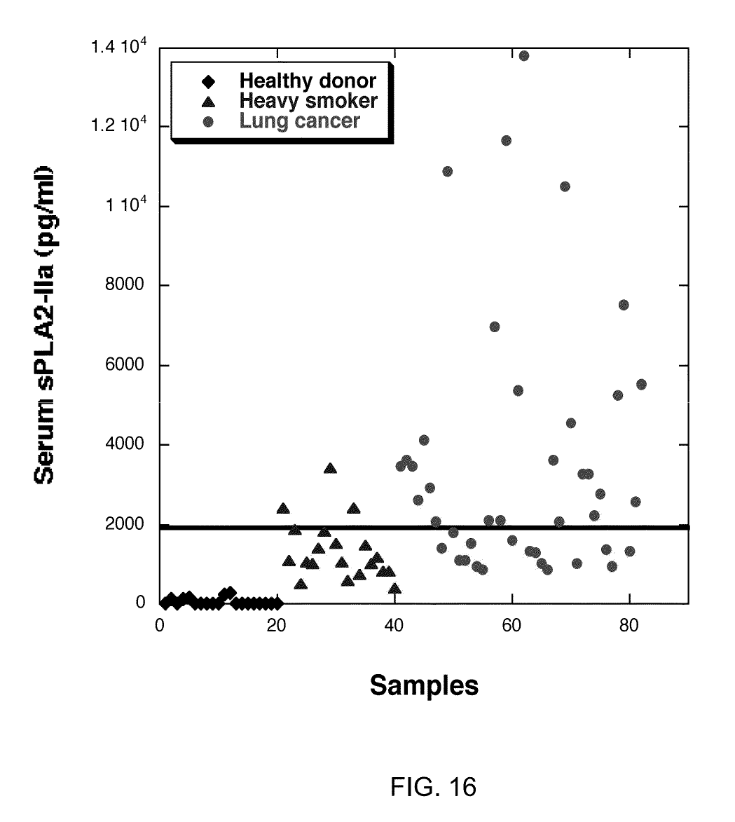

As shown in FIG. 16, the levels of serum sPLA2-IIA in plasma samples in lung cancer patients were significantly higher than those in heavy smokers without lung cancers (P=0.0002). Specifically, the levels of serum sPLA2-IIA ranged from about 0 pg/mL to about 245 pg/mL in healthy donors and age was not significantly associated with the levels of serum sPLA2-IIA in the normal cohort. ROC analysis resulted in 93% positive predictive value (hereinafter “PPV”) with a cutoff value of about 1.9 ng/mL. Moreover, as shown in Table 3 below, specificity and sensitivity of heavy smoker plasma samples and lung cancer plasma samples were 85% and 62% respectively.

TABLE 3
Heavy Smoker vs. Lung Cancer Cohort

| | Serum sPLA2-II | | |
|---|---|---|---|
| | <1.9 ng/ml | >1.9 ng/ml | Total case# |
| Heavy Smoker | 17 | 3 | 20 |
| Lung Cancer | 16 | 26 | 42 |

Cutoff value = 1.9 ng/ml
PPV = 93%
Specificity = 85%
Sensitivity = 62%

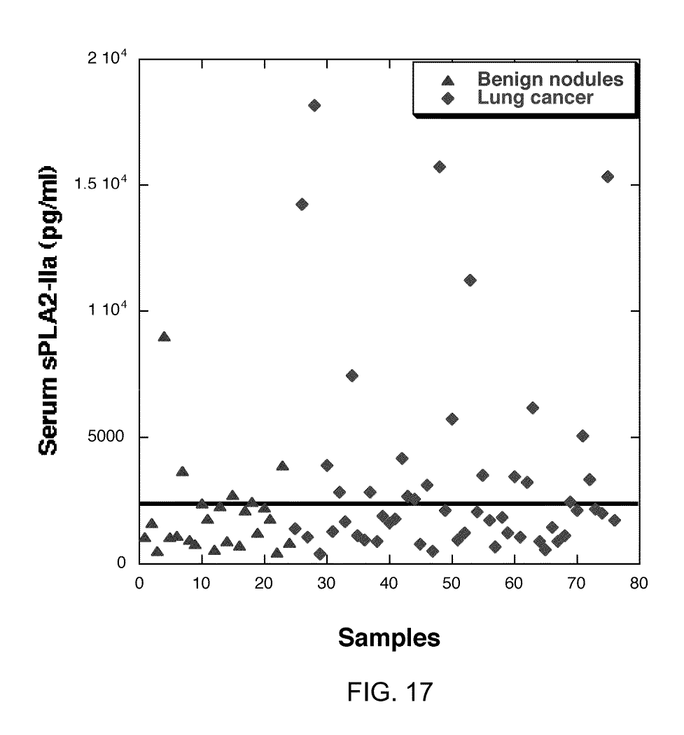

As shown in FIG. 17, the levels of serum sPLA2-IIA in plasma samples in lung cancer patients were significantly higher than those in patients with benign lung nodules (unpaired t test: P=0.028). ROC analysis resulted in 80% PPV with a cutoff value of 2.36 ng/mL. Moreover, as shown in Table 4 below, specificity and sensitivity of the benign nodule plasma samples and lung cancer plasma samples were 75% and 40%.

TABLE 4
Benign Lung Nodule vs. Lung Cancer Cohort

| | Serum sPLA2-IIa | | |
|---|---|---|---|
| | <2.36 ng/ml | >2.36 ng/ml | Total case# |
| Benign nodule | 18 | 6 | 24 |
| Lung cancer | 31 | 21 | 52 |

Cutoff value = 2.36 ng/ml
PPV = 80%
Specificity = 75%
Sensitivity = 40%

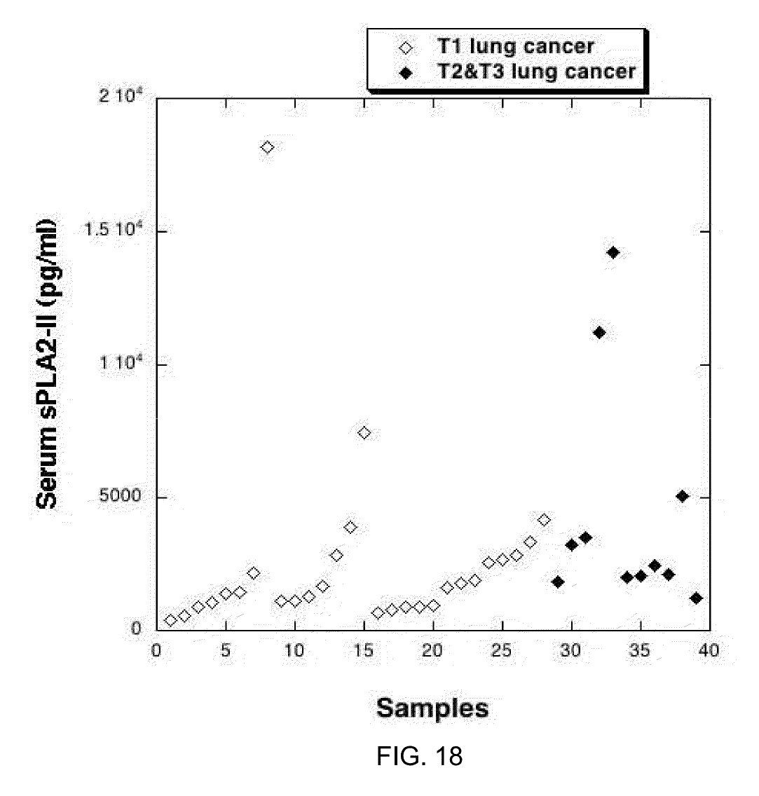

As shown in FIG. 18, the levels of serum sPLA2-IIA were significantly associated with T2 and T3 stage relative to the early T1 stage of lung cancer.

### Example 6

### Serum sPLA2-IIA, CEA, and Cyfra 21.1 are Elevated in Lung Cancer Patients

Experimental Protocol.

As part of the University of Cincinnati Thoracic Tumor Registry, plasma samples were collected pre-operatively from patients with pulmonary nodules known or suspected to be lung cancer undergoing resection. Data on the final pathology of the resected nodules was collected and defined as the benign nodule-lung cancer cohort (hereinafter “BNLCC”). Plasma samples were also collected from lung cancer patients from the Genetic Epidemiology of Lung Cancer cohort (hereinafter “GELCC”), a familial lung cancer cohort. Additionally, plasma samples from healthy donors were obtained from the Cincinnati Hoxworth Blood Center. This experiment was conducted in accordance with the Declaration of Helsinki and the local Institutional Review Board approved the experimental protocol.

sPLA2-IIA levels in the plasma samples were determined by diluting the plasma samples ten times and then subjecting them to duplicate ELISA analysis using the kit previously described. The average of the duplicate sample was calculated to present as pg/mL based on the standard curve of each experiment.

Given the heterogeneous nature of lung cancer, a combined blood test including a panel of biomarkers was predicted to increase the sensitivity for prediction of lung cancer. Accordingly, a combined blood test including sPLA2-IIA, CEA and Cyfra 21.1 was designed. sPLA2-IIA levels were determined as previously described. CEA and Cyfra 21.1 levels were determined via ELISA analysis and quantitated against a standard curve of each ELISA assay. More specifically, CEA levels were determined with EIA-1871 ELISA kit (DRG International Inc., Springfield, N.J.), and Cyfra 21.1 levels were determined with EIA-3943 ELISA kit (DRG International Inc.).

Immunohistochemistry analysis of sPLA2-IIA expression in an array of lung tissue specimens was also performed. Specifically, immunohistochemistry analysis of sPLA2-IIA expression was performed in the following lung tissue specimens: squamous cell carcinoma specimens, adenocarcinoma specimens, bronchioalveolar carcinoma specimens, small cell carcinoma specimens, metastatic squamous cell carcinoma specimens, atypical carcinoid (malignant tumor) specimens, inflammatory pseudo tumor specimens, normal lung tissue specimens, and SP-C/Tag mouse lung cancer specimens. Lung tissue specimens were obtained as a lung disease spectrum tissue array (Biomatrix US). Immunohistochemistry analysis was performed in accordance with the following procedure. Paraffin-embedded tissue sections were treated with xylene to deparaffinize. The tissue sections were then rehydrated in graded alcohol and transferred to PBS. The slides were then treated with a citric acid-based antigen-retrieval buffer (DAKO Co.), followed by 3% H₂O₂ in methanol, incubated in blocking buffer (5% BSA and 5% horse serum in PBS) and then in the blocking buffer containing the primary antibody. After washing, the slides were incubated with a biotinylated secondary antibody (BioGenex Laboratories), followed by washing and incubation with streptavidin-conjugated peroxidase (BioGenex). A positive reaction was visualized by incubating the slides with stable diaminobenzidine and counterstaining with Gill's hematoxylin (BioGenex) and mounted with Universal Mount mounting medium (Fisher Scientific). The intensity and extent of positive labeling for sPLA2-IIA in tissue arrays were assessed semiquantitatively and scored as 0 (no staining), 1+ (weak and focal staining in <25% of tissue), 2+ (moderate intensity in 25-50% of tissue), 3+ (moderate intensity in >50% of tissue) and 4+ (strong and diffused staining in >50% of tissue).

The means and standard deviations were calculated with confirmed significant differences in plasma sPLA2-IIA levels between lung cancer specimens and benign nodule specimens. Geometric means and standard deviations were also calculated with confirmed significant difference in plasma sPLA2-IIA levels between lung cancer specimens and clinical control specimens. Unpaired t-test with Welch correction was performed to evaluate the difference between sPLA2-IIA means of (i) 96 lung cancer specimens versus 29 benign SPN specimens from the BNLCC; (ii) 44 T1 stage (i.e., stage one) lung cancer specimens versus 18 T2 stage (i.e., stage two) lung cancer specimens from the BNLCC; (iii) 44 lung cancer specimens from the GELCC versus 29 benign SPN specimens from the BNLCC. Unpaired t test with Welch correction was also used to analyze overall cancer survival year associated with plasma sPLA2-IIA levels from the BNLCC.

A parametric Receiver Operating Characteristic (i.e., ROC) analysis of plasma sPLA2-IIA values was also performed to evaluate the ability of the levels of plasma sPLA2-IIA to distinguish 96 patients with lung cancer from 29 patients with benign SPNs from the BNLCC. 44 lung cancer specimens from the GELCC relative to 29 benign SPN specimens from the BNLCC were also subjected to the ROC analysis. The optimum cutoff value of plasma sPLA2-IIA was determined by calculating the Youden Index, which separated the combined set of sPLA2-IIA values into two groups, such that the number of correctly classified specimens was maximized, and the associated sensitivity and specificity for predicting lung cancer versus non-malignant nodule were determined.

Experimental Results.

Referencing Tables 5-8 below, the means and standard deviations of plasma sPLA2-IIA levels from 96 patients with lung cancer and 29 patients with benign lung nodules (i.e., SPNs) from the BNLCC were respectively 3646±407.3 pg/mL and 1772±306.8 pg/mL. Based on an unpaired t-test, the average plasma sPLA2-IIA levels in lung cancer patients was significantly higher than that in the non-malignancy SPN controls (P=0.004).

TABLE 5
Characteristics of Patients

| | |
|---|---|
| BNLCC-Lung cancer | 96 |
| M/F | 37/59 |
| Mean age year (range) | 64 (41~88) |
| NSCLC | 93 |
| Adenocarcinoma | 54 |
| Squamous carcinoma | 21 |
| Other NSCLC | 18 |
| SCLC | 3 |
| Stage | |
| I | 44 |
| II | 18 |
| III + IV | 4 |
| Blooddraw before resection | 96 |
| Blooddraw after resection | 0 |
| BNLCC-benign SPN | 29 |
| M/F | 15/14 |
| Mean age year (range) | 56 (31~81) |
| Blooddraw before resection | 29 |
| Blooddraw after resection | 0 |
| GELCC-Lung cancer | 44 |
| M/F | 18/26 |
| Mean age year (range) | 61 (47~70) |
| Healthy donor | 20 |
| Mean age year (range) | 49 (24~65) |

TABLE 6
Plasma sPLA2-IIA Levels and Diagnosis
in Patients with Benign SPNs from BNLCC

| | sPLA2-IIa | |
|---|---|---|
| Sample | (pg/ml) | Diagnosis |
| 1 | 1039.2 | 0.9 cm nodule and heavy smoker |
| 2 | 1589.2 | 1.3 × 1.4 × 1.1 cm subpleural nodule, necrotizing granuloma |
| 3 | 480.83 | Nodule 2 cm |
| 4 | 8980.8 | Necrotizing granulomatous |
| 5 | 1030.8 | Nonnecrotizing granulomas |
| 6 | 1105.8 | Abscess colonized with fungal hyphae |
| 7 | 3680.8 | Nodule with organizing pneumonia pattern of lung injury |
| 8 | 922.5 | Caseating granulomas with emphysematous changes |
| 9 | 797.5 | Nodules of caseating granulomas |
| 10 | 2372.5 | Necrotizing granuloma with fungal organisms |
| 11 | 1789.2 | Peribronchial tumorlet-2 mm and focal parenchymal fibrosis |
| 12 | 580.83 | Necrotizing granuloma with fungal organisms |
| 13 | 2305.8 | Necrotizing granuloma (2 × 1.7 × 1.3 cm) |
| 14 | 872.5 | Broncial and broncheolar ectasis with fibrosis and chronic |
| | | inflammation c/w bronchiectasis |
| 15 | 2714.2 | Bronchiolitis obliterans organizing pneumonia |
| 16 | 714.17 | Focal fibrosis and chronic inflammation and reactive pneumocyte |
| | | hyperplasia |
| 17 | 2105.8 | Caseating granulomas with fungal elements |
| 18 | 2422.17 | Myolipomatous polyp |
| 19 | 1226.52 | Active granulomatous inflammation and possible sarcoidosis |
| 20 | 2217.83 | Neurofibroma, 4.5 × 3.5 cm |
| 21 | 1804.78 | Active non-caseating granulomas |
| 22 | 430.87 | Necrotizing granuloma with fungal organisms |
| 23 | 3878.7 | Caseating necrotizing granulomas with fungal elements |
| 24 | 835.22 | Non-caseating granulomas with fungal elements |
| 25 | 572.5 | Necrotizing granulomatous inflammation with fungal organisms |
| | | consistent with Aspergillus |
| 26 | 1553.75 | Necrotizing granulomas with fungal yeast forms consistent with |
| | | Histoplasma |
| 27 | 816.25 | Organizing suppurative bronchopneumonia with pleural adhesions |
| 28 | 1328.75 | Presented with stable lung nodule |
| 29 | 1228.13 | necrotizing granuloma and Langerhans' cell histiocytosis |

*The data in bold is higher than the cutoff value of the blood test.

TABLE 7
Levels of Plasma sPLA2-IIA, Cyfra 21.1, and
CEA in Lung Cancer Patients from BNLCC

| | | | | | sPLA2-IIa | Cyfra21.1 | CEA | Survival |
|---|---|---|---|---|---|---|---|---|
| Sample | Stage | Diagnosis | Age | Sex | (pg/ml) | (ng/ml) | (ng/ml) | year |
| 1 | pT1 | Squamous cell | 65 | F | 2151.43 | 0 | 0 | |
| | | carcinoma | | | | | | |
| 2 | pT1 | Adenocarcinoma | 60 | F | 18189 | 0 | 0 | |
| 3 | pT1 | Adenocarcinoma | 81 | M | 415.71 | 1.63 | 0 | |
| 4 | pT1 | Squamous cell | 63 | M | 564.17 | 0 | 0.840 | 4 |
| | | carcinoma | | | | | | |
| 5 | pT1 | Squamous cell | 75 | F | 1422.5 | 0 | 1.690 | 4 |
| | | carcinoma | | | | | | |
| 6 | pT1 | Squamous cell | 81 | F | 883.04 | 0 | 2.535 | |
| | | carcinoma | | | | | | |
| 7 | pT1 | Adenocarcinoma | 61 | F | 1080.8 | 0 | 9.249 | |
| 8 | pT1 | Adenocarcinoma | 69 | M | 1414.2 | 0 | 13.286 | |
| 9 | pT1 | Adenocarcinoma | 79 | F | 3505.88 | 0 | 2.886 | |
| 10 | pT1A | Squamous cell | 52 | M | 1130.8 | 0 | 0 | |
| | | carcinoma | | | | | | |
| 11 | pT1A | Adenocarcinoma | 76 | F | 1139.57 | 0 | 0 | |
| 12 | pT1A | Adenocarcinoma | 79 | F | 1661.3 | 0 | 0 | |
| 13 | pT1A | Adenocarcinoma | 68 | M | 1753.13 | 0 | 0 | |
| 14 | pT1A | Adenocarcinoma | 63 | F | 2753.13 | 0 | 0 | |
| 15 | pT1A | Adenocarcinoma | 43 | F | 2814.2 | 0 | 0 | 4 |
| 16 | pT1A | Adenocarcinoma | 44 | F | 3914.2 | 0 | 0 | |
| 17 | pT1A | Adenocarcinoma | 45 | F | 3934.38 | 0 | 0 | |
| 18 | pT1A | Adenocarcinoma | 51 | M | 884.38 | 2.88 | 0 | |
| 19 | pT1A | Squamous cell | 66 | M | 5171.88 | 8.98 | 0 | |
| | | carcinoma | | | | | | |
| 20 | pT1A | Adenocarcinoma | 66 | M | 7439.57 | 18.7 | 0.469 | |
| 21 | pT1A | Adenocarcinoma | 71 | F | 1264.2 | 1.16 | 1.080 | |
| 22 | pT1A | Adenocarcinoma | 70 | F | 2784.38 | 8.45 | 29.353 | |
| 23 | pT1a | Adenocarcinoma | 48 | F | 1394.11 | 0 | 0 | |
| 24 | pT1a | Adenocarcinoma | 70 | M | 1452.94 | 0 | 0 | |
| 25 | pT1a | Adenocarcinoma | 62 | F | 3070.58 | 0 | 0 | |
| 26 | pT1a | Squamous cell | 65 | F | 5547.051 | 0 | 0 | |
| | | carcinoma | | | | | | |
| 27 | pT1a | Adenocarcinoma | 64 | F | 6288.23 | 0 | 0 | |
| 28 | pT1a | Squamous cell | 76 | F | 2300 | 3.33 | 0 | |
| | | carcinoma | | | | | | |
| 29 | pT1a | Adenocarcinoma | 76 | F | 12482.35 | 0 | 0 | |
| 30 | pT1b | Carcinoid tumor | 57 | F | 696.09 | 0 | 0 | |
| 31 | pT1B | Adenocarcinoma | 65 | F | 917.83 | 0 | 0 | |
| 32 | pT1B | Adenocarcinoma | 62 | F | 1604.78 | 0 | 0 | |
| 33 | pT1B | Adenocarcinoma | 68 | F | 2678.7 | 0 | 0 | |
| 34 | pT1B | Adenocarcinoma | 86 | F | 3759.38 | 0 | 0 | |
| 35 | pT1B | Adenocarcinoma | 65 | M | 2530.87 | 3.25 | 0 | |
| 36 | pT1B | Carcinoma, | 68 | M | 914.17 | 0 | 0.140 | |
| | | spindled cell type | | | | | | |
| 37 | pT1B | Adenocarcinoma | 63 | M | 4384.38 | 0 | 0.249 | |
| 38 | pT1B | Adenocarcinoma | 64 | F | 2830.8 | 0 | 0.563 | |
| 39 | pT1B | Adenocarcinoma | 62 | F | 1783.04 | 17.93 | 3.380 | |
| 40 | pT1B | Adenocarcinoma | 81 | F | 4170 | 0 | 4.930 | |
| 41 | pT1B | Squamous cell | 62 | M | 3348.26 | 1.35 | 5.023 | 1 |
| | | carcinoma | | | | | | |
| 42 | pT1B | Adenocarcinoma | 54 | F | 1874.35 | 0.53 | 5.446 | 2 |
| 43 | pT1B | Adenocarcinoma | 62 | F | 939.17 | 0 | 12.911 | |
| 44 | pT1B | Adenocarcinoma | 48 | F | 804.78 | 0 | 14.836 | |
| 45 | pT2 | Neuroendocrine | 88 | M | 1822.5 | 0 | 0 | |
| | | carcinoma | | | | | | |
| 46 | pT2 | Small cell | 65 | F | 3251.43 | 0 | 0 | |
| | | carcinoma | | | | | | |
| 47 | pT2 | NSCLC | 55 | M | 3501.43 | 0 | 0 | |
| 48 | pT2 | NSCLC | 56 | M | 14223 | 0 | 0 | 3 |
| 49 | pT2 | Adenosquamous | 76 | F | 11239.57 | 0 | 0.235 | |
| | | Carcinoma | | | | | | |
| 50 | pT2 | Squamous cell | 54 | M | 8385 | 0 | 1.315 | |
| | | carcinoma | | | | | | |
| 51 | pT2 | Squamous cell | 67 | F | 4266.25 | 0 | 2.160 | |
| | | carcinoma | | | | | | |
| 52 | pT2A | adenocarcinoma | 65 | F | 1828.13 | 0 | 8.408 | |
| 53 | pT2a | adenocarcinoma | 60 | F | 1782.35 | 0 | 0 | |
| 54 | pT2a | NSCLC | 68 | F | 5358.82 | 0 | 0.796 | |
| 55 | pT2a | Adenocarcinoma | 79 | M | 1941.17 | 0 | 0 | |
| 56 | pT2B | Adenocarcinoma | 64 | F | 4078.13 | 0 | 0 | |
| 57 | pT2B | Squamous cell | 43 | M | 17309.38 | 7.67 | 0 | |
| | | carcinoma | | | | | | |
| 58 | pT2B | NSCLC | 77 | F | 2048.26 | 0 | 1.690 | |
| 59 | pT2B | Squamous cell | 66 | M | 2026.52 | 0 | 4.225 | 5 |
| | | carcinoma | | | | | | |
| 60 | pT2B | Squamous cell | 74 | F | 2430.8 | 0 | 7.277 | |
| 61 | | carcinoma | | | | | | |
| | pT2B | adenocarcinoma | 60 | F | 13603.13 | 16.71 | 102.413 | |
| 62 | pT2b | adenocarcinoma | 76 | F | 2458.82 | 0 | 0 | |
| 63 | pT3 | Squamous cell | 63 | F | 2089.2 | 0.66 | 0 | 1 |
| | | carcinoma | | | | | | |
| 64 | pT3 | Squamous cell | 69 | F | 5039.2 | 4.33 | 0 | |
| | | carcinoma | | | | | | |
| 65 | pT3B | NSCLC | 56 | M | 1209.13 | 0 | 0 | 2 |
| 66 | pT4 | Squamous cell | 57 | M | 1717.83 | 0 | 21.221 | |
| | | carcinoma | | | | | | |
| 67 | | Squamous cell | 67 | M | 397.5 | 0 | 0 | 2 |
| | | carcinoma | | | | | | |
| 68 | | Adenocarcinoma | 62 | M | 505.83 | 0 | 0 | |
| 69 | | Adenocarcinoma | 76 | F | 622.5 | 0 | 0 | |
| 70 | | Adenocarcinoma | 63 | M | 685 | 0 | 0 | |
| 71 | | Small cell | 60 | F | 1044.29 | 0 | 0 | |
| | | carcinoma | | | | | | |
| 72 | | Adenocarcinoma | 44 | F | 1239.2 | 0 | 0 | 3 |
| 73 | | Squamous cell | 63 | M | 1278.75 | 0 | 0 | |
| | | carcinoma | | | | | | |
| 74 | | Adenocarcinoma | 58 | F | 2087.14 | 0 | 0 | 1 |
| 75 | | Squamous cell | 72 | M | 2803.75 | 0 | 0 | 1 |
| | | carcinoma | | | | | | |
| 76 | | Neuroendocrine | 75 | F | 3465.71 | 0 | 0 | |
| | | w/brain mets | | | | | | |
| 77 | | NSCLC | 41 | F | 4510 | 0 | 0 | |
| 78 | | NSCLC | 58 | M | 14066.25 | 0 | 0 | 1 |
| 79 | | Squamous cell | 87 | M | 15351.43 | 0 | 0 | |
| | | carcinoma | | | | | | |
| 80 | | Adenocarcinoma | 73 | M | 5735.22 | 3.6 | 0 | 1 |
| 81 | | NSCLC | 53 | F | 5310 | 33.3 | 0 | |
| 82 | | Adenocacinoma | 52 | F | 1635 | 0 | 0.423 | 5 |
| 83 | | NSCLC | 73 | M | 447.5 | 0 | 1.174 | |
| 84 | | Adenocarcinoma | 66 | F | 3130.8 | 0 | 3.474 | |
| 85 | | NSCLC | 52 | M | 2760 | 0 | 4.366 | |
| 86 | | Adenocarcinoma | 62 | M | 1897.5 | 0 | 5.399 | |
| 87 | | NSCLC | 67 | M | 1478.75 | 0 | 7.960 | |
| 88 | | Bronchial cancer | 58 | F | 1722.5 | 0 | 9.296 | |
| 89 | | NSCLC | 84 | F | 735 | 3.44 | 13.380 | 7 |
| 90 | | Adenocarcinoma | 67 | M | 3235 | 0 | 15.352 | |
| 91 | | Adenocarcinoma | 55 | F | 491.25 | 0 | 29.014 | |
| 92 | | Small cell | 58 | F | 6187.14 | 0 | 58.209 | |
| | | carcinoma | | | | | | |
| 93 | | Adenocarcinoma | 45 | M | 772.5 | 4.96 | 68.451 | |
| 94 | | Adenocarcinoma | 70 | M | 972.5 | 0 | 70.939 | |
| 95 | | Adenocarcinoma | 45 | F | 15715.71 | 0 | 71.866 | |
| 96 | | NSCLC | 62 | F | 6060 | 175.56 | 201.268 | |

*The data in bold is higher than the cutoff value of plasma sPLA2-IIa, Cyfra21.1, and CEA.

TABLE 8
Plasma sPLA2-IIA, Cyfra 21.1, and CEA Levels
and Diagnosis with Benign SPNs from BNLCC

| | | | sPLA2-IIa | Cyfra21.1 | CEA | |
|---|---|---|---|---|---|---|
| Sample | Age | Sex | (pg/ml) | (ng/ml) | (ng/ml) | Diagnosis |
| 1 | 57 | M | 1039.2 | 0 | 0.000 | 0.9 cm nodule and heavy smoker |
| 2 | 40 | F | 1589.2 | 0 | 2.207 | 1.3 × 1.4 × 1.1 cm subpleural nodule, |
| | | | | | | necrotizing granuloma |
| 3 | 55 | M | 480.83 | 0 | 0.000 | Nodule 2 cm |
| 4 | 58 | F | 8980.8 | 2.3 | 0.000 | Necrotizing granulomatous |
| 5 | 47 | F | 1030.8 | 0 | 1.127 | Nonnecrotizing granulomas |
| 6 | 71 | F | 1105.8 | 0 | 0.986 | Abscess colonized with fungal hyphae |
| 7 | 44 | F | 3680.8 | 0 | 0.000 | Nodule with organizing pneumonia pattern |
| | | | | | | of lung injury |
| 8 | 73 | M | 922.5 | 0 | 0.798 | Caseating granulomas with |
| | | | | | | emphysematous changes |
| 9 | 57 | M | 797.5 | 0 | 0.094 | Nodules of caseating granulomas |
| 10 | 48 | F | 2372.5 | 0 | 3.239 | Necrotizing granuloma with fungal |
| | | | | | | organisms |
| 11 | 72 | M | 1789.2 | 0 | 0.000 | Peribronchial tumorlet-2 mm and focal |
| | | | | | | parenchymal fibrosis |
| 12 | 36 | F | 580.83 | 0 | 0.000 | Necrotizing granuloma with fungal |
| | | | | | | organisms |
| 13 | 53 | M | 2305.8 | 0 | 12.582 | Necrotizing granuloma (2 × 1.7 × 1.3 cm) |
| 14 | 49 | F | 872.5 | 0 | 1.784 | Broncial and broncheolar ectasis with |
| | | | | | | fibrosis and chronic inflammation c/w |
| | | | | | | bronchiectasis |
| 15 | 52 | M | 2714.2 | 0 | 1.737 | Bronchiolitis obliterans organizing |
| | | | | | | pneumonia |
| 16 | 48 | M | 714.17 | 0 | 0.000 | Focal fibrosis and chronic inflammation |
| | | | | | | and reactive pneumocyte hyperplasia |
| 17 | 52 | M | 2105.8 | 0 | 0.000 | Caseating granulomas with fungal |
| | | | | | | elements |
| 18 | 48 | M | 2422.17 | 14.9 | 0.000 | Myolipomatous polyp |
| 19 | 64 | F | 1226.52 | 0 | 0.000 | Active granulomatous inflammation and |
| | | | | | | possible sarcoidosis |
| 20 | 31 | F | 2217.83 | 0 | 0.000 | Neurofibroma, 4.5 × 3.5 cm |
| 21 | 72 | F | 1804.78 | 0.22 | 0.000 | Active non-caseating granulomas |
| 22 | 46 | M | 430.87 | 0 | 0.000 | Necrotizing granuloma with fungal |
| | | | | | | organisms |
| 23 | 72 | F | 3878.7 | 0 | 0.000 | Caseating necrotizing granulomas with |
| | | | | | | fungal elements |
| 24 | 67 | F | 835.22 | 0 | 0.000 | Non-caseating granulomas with fungal |
| | | | | | | elements |
| 25 | 65 | M | 572.5 | 0 | 0.000 | Necrotizing granulomatous inflammation |
| | | | | | | with fungal organisms consistent with |
| | | | | | | Aspergillus |
| 26 | 81 | M | 1553.75 | 0 | 0.000 | Necrotizing granulomas with fungal yeast |
| | | | | | | forms consistent with Histoplasma |
| 27 | 46 | M | 816.25 | 0 | 0.100 | Organizing suppurative |
| | | | | | | bronchopneumonia with pleural adhesions |
| 28 | 72 | M | 1328.75 | 8.32 | 0.000 | Presented with lung nodule. Three years |
| | | | | | | post blood draw, the nodule remained |
| | | | | | | stable in size |
| 29 | 53 | F | 1228.13 | 0 | 1.194 | necrotizing granuloma and Langerhans' |
| | | | | | | cell histiocytosis |

*The data in bold is higher than the cutoff value of the blood test.

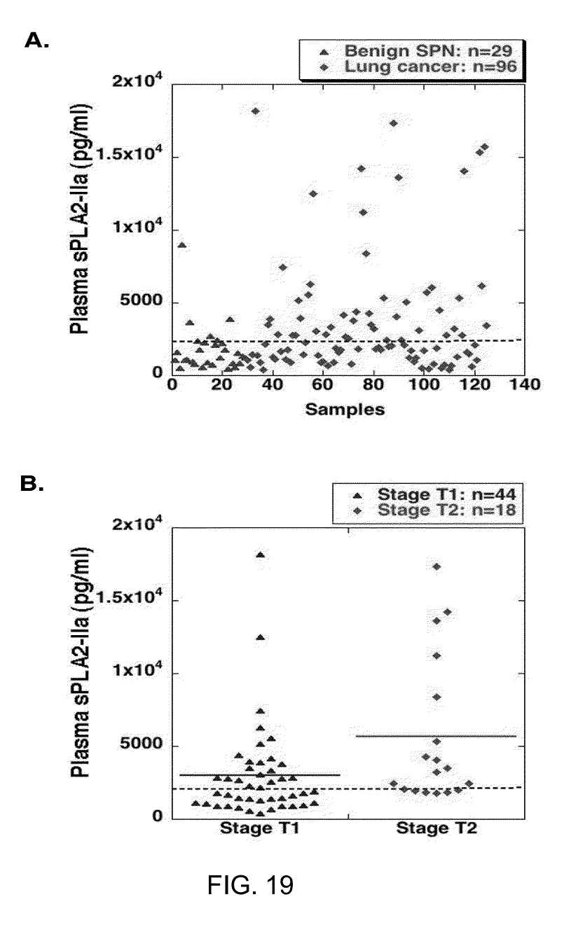

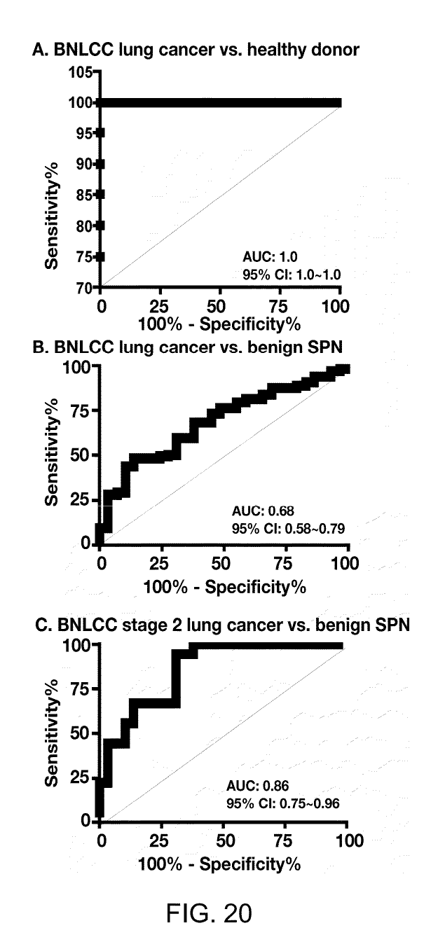

Referencing FIG. 19(A) and Table 9 below, the optimum cutoff value of plasma sPLA2-IIA by ROC analysis was 2.4 ng/mL, which resulted in 48% sensitivity and 86% specificity for predicting the presence of lung cancer. As shown in FIG. 20(B), the area under the curve (hereinafter “AUC”) was 0.68 (95% CI: 0.58˜0.79).

TABLE 9
High Level of Plasma sPLA2-IIA Predicts Lung Cancer

| | | | | | |
|---|---|---|---|---|---|
| Plasma sPLA2-IIa | <2.4 ng/ml | >2.4 ng/ml | Total case# | % Sensitivity | % Specificity |

BNLCC lung cancer vs. benign SPN

| | | | | | |
|---|---|---|---|---|---|
| Benign SPN | 24 | 5 | 29 | | 86 |
| Lung cancer | 50 | 46 | 96 | 48 | |
| Stage 1 lung cancer | 24 | 20 | 44 | 45 | |
| Stage 2 lung cancer | 6 | 12 | 18 | 67 | |

GELCC lung cancer vs. benign SPN

| Lung cancer | 16 | 28 | 44 | 64 | |
|---|---|---|---|---|---|
| sPLA2-IIa | <2.4 ng/ml | >2.4 ng/ml | | | |
| CEA | <6.0 ng/ml | >6.0 ng/ml | | | |
| Cyfra21.1 | <3.3 ng/ml | >3.3 ng/ml | Total case# | % Senstivity | % Specificity |

BNLCC lung cancer vs. benign SPN

| Benign SPN | 22 | 7 | 29 | | 76 |
|---|---|---|---|---|---|
| Lung cancer | 36 | 60 | 96 | 63 | |
| Stage 1 lung cancer | 18 | 26 | 44 | 59 | |
| Stage 2 lung cancer | 5 | 13 | 18 | 72 | |
| CEA | <6.0 ng/ml | >6.0 ng/ml | Total case# | % Senstivity | % Specificity |

BNLCC lung cancer vs. benign SPN

| Benign SPN | 28 | 1 | 29 | | 97 |
|---|---|---|---|---|---|
| Lung cancer | 77 | 19 | 96 | 20 | |
| Cyfra21.1 | <3.3 ng/ml | >3.3 ng/ml | Total case# | % Senstivity | % Specificity |

BNLCC lung cancer vs. benign SPN

| | | | | | |
|---|---|---|---|---|---|
| Benign SPN | 27 | 2 | 29 | | 93 |
| Lung cancer | 83 | 13 | 96 | 14 | |

The means and standard deviations of plasma sPLA2-IIA levels from 44 lung cancer samples from the GELCC were 3718±407.8. The average plasma sPLA2-IIA level in these lung cancer patients was also significantly higher than that in patients with benign SPNs from the BNLCC (P=0.003). Referencing Table 9, the optimum cutoff value of plasma sPLA2-IIA by ROC analysis was 2.4 ng/mL, which resulted in 64% sensitivity and 86% specificity for predicting lung cancer (AUC: 0.79; CI: 0.69˜0.90).

On the other hand, referencing Table 10 below, among 20 healthy donors, the levels of plasma sPLA2-IIA were undetectable in 15 healthy donors, while those of the remaining 5 donors ranged up to 275 pg/mL. Referencing Table 5, age was not significantly associated with the level of plasma sPLA2-IIA in this healthy donor cohort. As shown in FIG. 20(A), ROC analysis of the plasma sPLA2-IIA levels in 96 lung cancer samples from the BNLCC and 20 healthy donor samples revealed an AUC as 1.0 (95% CI: 1.0˜2.0).

TABLE 10
Plasma sPLA2-IIA Levels in Healthy Donors

| Plasma | sPLA2-IIa | |
|---|---|---|
| Samples | (pg/ml) | Age |
| Healthy 1 | 0.00 | 53 |
| Healthy 2 | 115.91 | 34 |
| Healthy 3 | 0.00 | 48 |
| Healthy 4 | 111.36 | 62 |
| Healthy 5 | 145.45 | 60 |
| Healthy 6 | 0.00 | 55 |
| Healthy 7 | 0.00 | 46 |
| Healthy 8 | 0.00 | 29 |
| Healthy 9 | 0.00 | 64 |
| Healthy 10 | 0.00 | 52 |
| Healthy 11 | 217.73 | 64 |
| Healthy 12 | 275.45 | 65 |
| Healthy 13 | 0.00 | 61 |
| Healthy 14 | 0.00 | 24 |
| Healthy 15 | 0.00 | 39 |
| Healthy 16 | 0.00 | 43 |
| Healthy 17 | 0.00 | 55 |
| Healthy 18 | 0.00 | 39 |
| Healthy 19 | 0.00 | 48 |
| Healthy 20 | 0.00 | 47 |

As shown in FIG. 19(B) and Table 7, plasma sPLA2-IIA was significantly higher in T2 stage lung cancer (n=18) relative to T1 stage lung cancer (n=44) from the BNLCC (Two tailed t test: P=0.005). Referencing Table 9 and FIG. 20(C), the optimum cutoff value of plasma sPLA2-IIA by ROC analysis was 2.4 ng/mL, which resulted in 67% sensitivity for predicting T2 stage lung cancer (AUC: 0.86; CI: 0.65˜0.96), as compared with 45% sensitivity for predicting T1 stage lung cancer. Referencing Table 7, high levels of plasma sPLA2-IIA were also associated with a decreased overall survival (unpaired t test with Welch correction: P=0.0457). The mean overall survival year for high sPLA2-IIA (>2.4 ng/mL, n=6) was 1.8±1.3 years, while the mean overall survival year for low sPLA2-IIA (<2.4 ng/mL, n=11) was 3.3±1.9 years.

Referencing Tables 7-9, it was discovered that a combination of sPLA2-IIA (2.4 ng/mL cutoff value), Cyfra 21.1 (3.3 ng/mL cutoff value) and CEA (6 ng/mL cutoff value) tests increased the sensitivity for lung cancer prediction up to 62% from 48% by sPLA2-IIA test alone for 96 cancers relative to 29 patients with benign SPNs from the BNLCC. Positive prediction for the presence of lung cancer by the combined blood tests was defined as one or more biomarkers higher than their cutoff value. Furthermore, the combined tests increased the sensitivity for lung cancer prediction up to 59% and 72% from 45% and 67% by sPLA2-IIA test alone for 44 T1 stage cancers and 18 T2 stage cancers relative to 29 patients with benign SPNs, respectively. As shown in Table 9, CEA and Cyfra 21.1 tests alone had only 20% and 14% sensitivity, respectively.

Additionally, referencing Table 6, among the 5 patients with benign lung nodules and higher level of plasma sPLA2-IIA than the cutoff value of 2.4 ng/mL, two patients suffered from localized pneumonia, one patient suffered from myolipomatous polyp, and the remaining two patients were diagnosed with necrotizing granulomas. Without being bound by the theory, these findings indicated that active localized inflammation can occasionally lead to a moderate increase in plasma sPLA2-IIA. Furthermore, the data demonstrated that there was an increased basal level of plasma sPLA2-IIA in patients with SPNs relative to those in healthy donors, which may result from SPNs and chronic inflammation. Moreover, such data indicated that plasma sPLA2-IIA can serve as a biomarker to predict more than 48% of T1 stage lung cancers and up to 67% of T2 stage lung cancers relative to patients with benign SPNs, although the plasma sPLA2-IIA is elevated occasionally in patients with benign SPNs. Additionally, the combined sPLA2-IIA, Cyfra 21.1, and CEA blood test increased the sensitivity for lung cancer prediction relative to spLA2-IIA testing alone. In addition, plasma sPLA2-IIA may potentially serve as a poor prognosis marker for lung cancer.

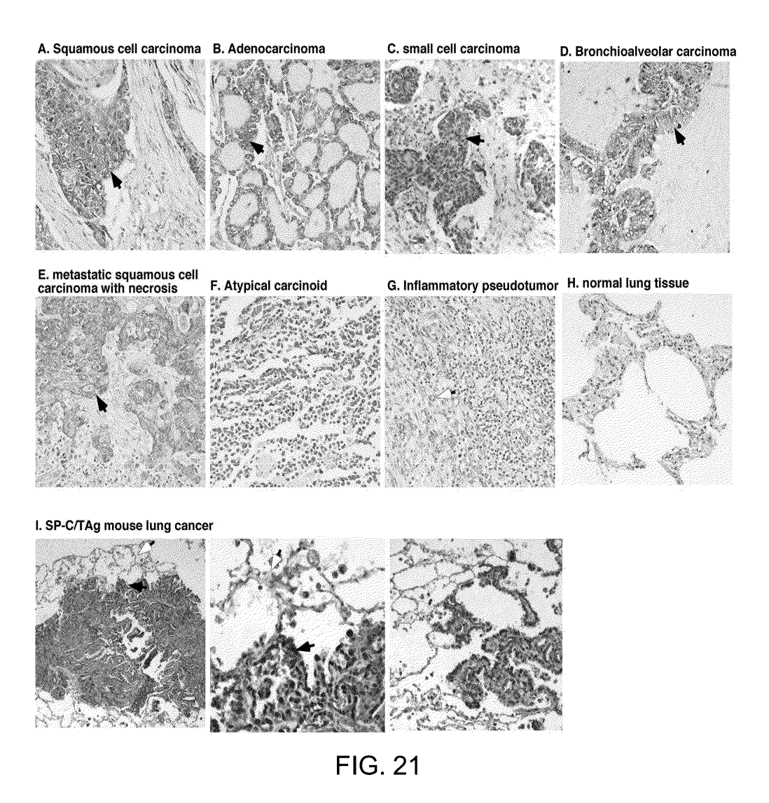

As shown in FIG. 21, among 100 core lung biopsies examined, sPLA2-IIA was overexpressed in 100% of squamous cell carcinoma (20 cores), 100% of adenocarcinoma (20 cores), and 100% of bronchioalveolar carcinoma (10 cores). sPLA2-IIA was also overexpressed in 70% of small cell carcinoma (10 cores) and in 90% of metastatic squamous cell carcinoma (10 cores). sPLA2-IIA was not detected in atypical carcinoid (malignant tumor, 5 cores), inflammatory pseudo tumor (5 cores), and normal lung tissue (15 cores). Moreover, IHC analysis demonstrated that moderate increased serum sPLA2-IIA in inflammation was due to expression of sPLA2-IIA by infiltrated macrophage and endothelial cells in new blood vessels of the inflammation site. Without being bound by the theory, expression of sPLA2-IIA in infiltrating macrophages and endothelial cells of new blood vessels in inflammatory pseudo tumors implicated an underlying mechanism of localized inflammation as the cause of occasional elevated levels of plasma spLA2-IIA in patients with benign lesions. Moreover, it was also discovered that spLA2-IIA was overexpressed in lung cancer tissue, but not in adjacent normal type I and II epithelial cells in the spontaneous mouse lung cancer from SP-C/Tag transgenic mice, in which the transgene SV40 early region (Tag) gene was driven by a 3.7 kb promoter of human surfactant protein C(SP-C) gene.

### Example 7

### sPLA2-IIA is a Potential Ligand of HER Family Receptors

Experimental Protocol.

A549 and H1975 lung cancer cells were starved in 1% stripped medium for 24 H. The cells were then treated with recombinant human sPLA2-IIA (0.5 μg/mL) with various concentrations (0, 0.125, 0.25, or 0.5 μg/mL) of sPLA2-IIA for 2 H in medium containing 10% fetal bovine serum (hereinafter “FBS”). Cell extracts were prepared and subjected to western blot analysis for HER2, P-HER2, HER3, and P-HER3. Western blot analysis was conducted as previously described.

Additionally, LNCaP-AI prostate cancer cells were starved in 1% stripped medium for 24 H. The cells were then treated with recombinant human sPLA2-IIA (0.5 μg/mL) for 30 to 180 minutes (0, 30, 60, 120, or 180 min) or with various concentrations of sPLA2-IIA (0, 0.125, 0.25, or 0.5 μg/mL) for 2 H in medium containing 10% FBS. Cell extracts were prepared and subjected to western blot analysis for P-HER2 and HER2. Western blot analysis was conducted as previously described.

Experimental Results.

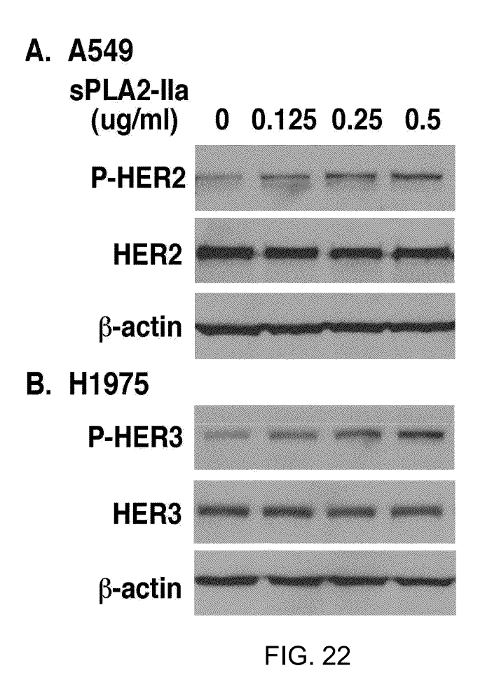

As shown in FIG. 22(A)-(B), sPLA2-IIA activates HER family receptors in A549 and H1975 lung cancer cells. More particularly, treatment of A549 and H1975 cells with recombinant human sPLA2-IIA induced phosphorylation of HER2 and HER3 in a dose-dependent manner.

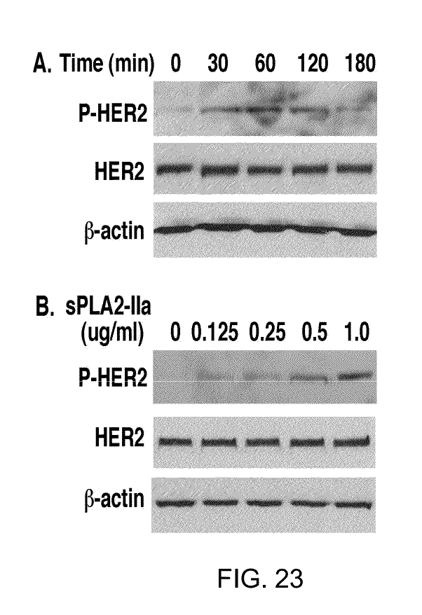

Additionally, as shown in FIGS. 23(A)-(B), sPLA2-IIA activates HER family receptors in LNCaP-AI prostate cancer cells. More particularly, treatment of LNCaP-AI cells with recombinant human sPLA2-IIA induced phosphorylation of HER2 in a time- and dose-dependent manner.

### Example 8

### High Levels of Plasma sPLA2-IIA are Associated with Advanced Prostate Cancer Stages

Experimental Protocol.

A total of 134 prostate cancer patients from the University Hospital (Cincinnati, Ohio) were consented, and subsequently plasma and tissue specimens and patients' medical information were obtained from University of Cincinnati Cancer Center Tissue Bank. sPLA2-IIA levels in plasma was determined by ELISA kit as previously described. sPLA2-IIA levels in the plasma samples were determined by diluting the plasma samples ten times and then subjecting them to duplicate ELISA analysis using the kit previously described. The average of the duplicate sample was calculated to present as pg/mL based on the standard curve of each experiment.

Unpaired t-test was performed to evaluate the difference of the mean levels of plasma sPLA2-IIA between stage T1 prostate cancer (n=18) and the stage T2-T4 prostate cancer (n=101) as well as between Gleason scores 6-7 (n=85) and Gleason scores 8-10 (n=41) prostate cancer. A parametric ROC analysis was performed to associate a high level of plasma sPLA2-IIA with advanced stage T2-T4 prostate cancer (n=101) relative to early stage T1 cancer (n=18) and with high Gleason scores 8-10 (n=41) prostate cancer relative to intermediate Gleason score 6-7 (n=85) cancer. The optimum cutoff value of plasma sPLA2-IIA was determined, which separated the combined set of sPLA2-IIA values into two groups, such that the number of correctly classified specimens was maximized, and the associated sensitivity, specificity, and AUC were determined.

Experimental Results.

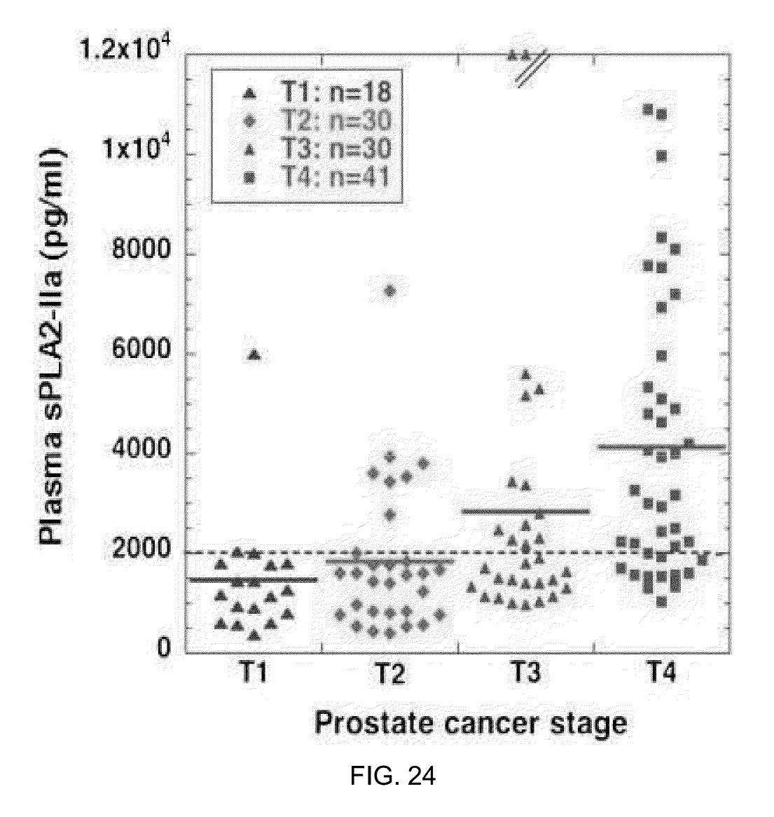

The mean levels of plasma sPLA2-IIA were 1479±1246, 1843±1481, 3130±3892, and 4141±1246 pg/mL for stage T1 (n=18), T2 (n=30), T3 (n=30), and T4 (n=41) prostate cancers, respectively. As shown in FIG. 24, levels of plasma sPLA2-IIA increased with prostate cancer progression.

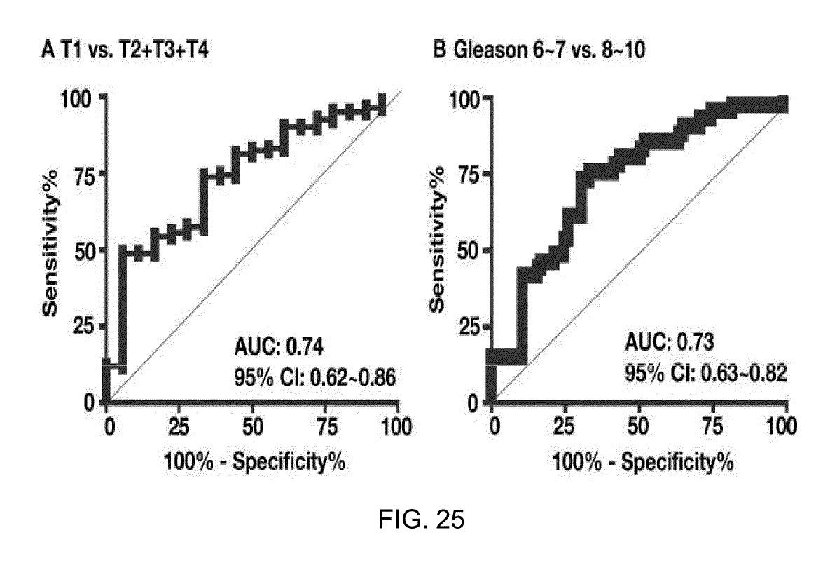

It was also determined that high levels of plasma sPLA2-IIA were associated with advanced cancer relative to indolent cancer by the ROC analysis. The indolent prostate cancer was defined as stage T1 and Gleason score 6 in this prostate cancer cohort. The mean value of plasma sPLA2-IIA values from the indolent stage T1 prostate cancer (n=18) and the advanced stage T2-T4 prostate cancer (n=101) were 1480±293 (n=18) and 3159±299 (n=101) pg/mL, respectively. The levels of plasma sPLA2-IIA from patients with advanced prostate cancer were significantly higher than those with indolent cancer (P<0.0001, unpaired t-test). As shown in FIG. 25(A), ROC analysis revealed that an optimum cutoff value for plasma sPLA2-IIA of 2.0 ng/mL predicted an advanced prostate cancer with 50% sensitivity and 83% specificity. AUC was 0.74 (95% confidence interval: 0.6204-0.8559). The mean ages for stage T1, T2, T3, and T4 prostate cancers were 69.28±9.59, 63.23±6.27, 62.67±8.94, and 65.56±11.22, respectively. As set forth in Table 11 below, the age of the patients was not significantly associated with the advanced stage of prostate cancer (P>0.2) and did not contribute to elevated plasma sPLA2-IIA.

TABLE 11
Characteristics of Patients

| Characteristics | Age | Number of patients |
|---|---|---|
| Prostate cancer | | 134 |
| Mean age year (range) | 65.7 (45~93) | |
| Stage | | |
| I | | 18 |
| II | | 30 |
| III | | 30 |
| IV | | 41 |
| Gleason score 6~7 | | 85 |
| Gleason score 8~10 | | 41 |
| Pretreatment samples | | 6 |
| Treatment samples | | 128 |

Further analysis demonstrated that the mean plasma sPLA2-IIA values from the intermediate Gleason scores (6-7; n=85) and high Gleason scores (8-10; n=41) prostate cancers were 2098±196 pg/mL and 4063±595 pg/mL, respectively. The levels of plasma sPLA2-IIA were significantly higher in prostate cancers with Gleason scores 8-10 than those with Gleason score 6-7 regardless of treatment (P<0.0001, unpaired t-test). As shown in FIG. 25(B), ROC analysis revealed that an optimum, cutoff value for plasma sPLA2-IIA of 2.0 ng/mL predicted prostate cancer of high Gleason score with 61% sensitivity and 73% specificity. The AUC was 0.73 (95% CI: 0.63-0.82). The mean ages for prostate cancer patients with intermediate Gleason scores and high Gleason scores were 66.98±10.18 and 63.41±9.43, respectively. Therefore, the age of the patients was not significantly associated with high Gleason score (P>0.1) and did not contribute to an elevated plasma sPLA2-IIA.

It was noteworthy that there was no significant correlation between high PSA levels and Gleason scores or cancer stage, since there was a wide range of PSA levels from 0.1 to 3300 ng/mL among 134 samples. Almost all 134 prostate cancer patients had been treated with a variety of modalities including hormone ablation therapy, radiotherapy, and prostatectomy. It should be recognized that these treatments significantly alter patient's plasma PSA levels. Nevertheless, the data strongly suggests that high levels of plasma sPLA2-IIA are associated with advanced cancer stage and high Gleason score and as such, represent a robust prognostic biomarker for the identification of poor prognosis prostate cancer.

### Example 9

### Combined Serum sPLA2-IIA, CEA, Cyfra 21.1, SCCA, and NSE Lung Cancer Protein Biomarker Blood Test

Experimental Protocol.

Given the heterogeneous nature of lung cancer, the sensitivity of a combined blood test (as set forth in Table 12 below) is investigated. Such combined blood test includes a panel of sPLA2-IIA, CEA, Cyfra 21.1, squamous cell carcinoma antigen (i.e., SCCA), and NSE biomarkers. Such panel of biomarkers may increase the sensitivity for prediction of lung cancer. More particularly, such panel of biomarkers may increase the sensitivity for prediction of early stage lung cancer.

TABLE 12
Combined Lung Cancer Protein Biomarker Blood Test

| | | | Cutoff | Preferable |
|---|---|---|---|---|
| Plasma | % Sensi- | % Speci- | Value | Histological |
| Biomarker | tivity | ficity | (ng/mL) | Types |
| sPLA2-IIA | 48 | 86 | 2.4 | All |
| Cyfra21.1 | 40 | 95 | 3.3 | Squamous Cell |
| | | | | Carcinoma |
| SCCA | 32 | 95 | 2.0 | Squamous Cell |
| | | | | Carcinoma |
| CEA | 25 | 95 | 6.0 | Adenocarcinoma |
| NSE | 32 | 95 | 12.5 | Small Cell |
| | | | | Lung Cancer |

As part of the University of Cincinnati Thoracic Tumor Registry, plasma samples are collected pre-operatively from patients with pulmonary nodules known or suspected to be lung cancer undergoing resection. Data on the final pathology of the resected nodules are collected and defined as the benign nodule-lung cancer cohort (hereinafter “BNLCC”). Plasma samples are collected from lung cancer patients from the Genetic Epidemiology of Lung Cancer cohort (hereinafter “GELCC”), a familial lung cancer cohort. Additionally, plasma samples from healthy donors are obtained from the Cincinnati Hoxworth Blood Center. This experiment is conducted in accordance with the Declaration of Helsinki and the local Institutional Review Board approved the experimental protocol.

sPLA2-IIA levels in the plasma samples are determined by diluting the plasma samples ten times and then subjecting them to duplicate ELISA analysis using the kit previously described. The average of the duplicate sample is calculated to present as pg/mL based on the standard curve of each experiment. CEA and Cyfra 21.1 levels are determined via ELISA analysis and quantitated against a standard curve of each ELISA assay as previously described. Additionally, SCCA and NSE levels are determined via ELISA analysis.

If the sensitivity of the combined blood test set forth in Table 12 is low, (which is unexpected), a combination of six lung cancer protein biomarkers are employed in a further combined blood test. More particularly, a combined blood test is designed to include a panel of sPLA2-IIA, CEA, Cyfra 21.1, SCCA, NSE, and progastrin releasing peptide (i.e., ProGRP). The preferable histological type of ProGRP is SCLC.

Experimental Results.

A combination of sPLA2-IIA (2.4 ng/mL cutoff value), Cyfra 21.1 (3.3 ng/mL cutoff value), CEA (6 ng/mL cutoff value), SCCA (2.0 ng/mL cutoff value), and NSE (12.5 ng/mL cutoff value) biomarkers may have a combined sensitivity of 89.2% based on specificity of 86-95%. Such prediction is based on an assumption of “average” correlation among the biomarkers, such that the numbers of true positives and false negatives for each biomarker are distributed in equal proportions on each cross tabulation.

A combination of sPLA2-IIA (2.4 ng/mL cutoff value), Cyfra 21.1 (3.3 ng/mL cutoff value), CEA (6 ng/mL cutoff value), SCCA (2.0 ng/mL cutoff value), NSE (12.5 ng/mL cutoff value), and ProGRP (300 pg/mL cutoff value) biomarkers may have a combined sensitivity of 91.5% based on specificity of 86-95%.

### Example 10

### Managing a Treatment Protocol of Prostate Cancer

Experimental Protocol.

As high levels of sPLA2-IIA are associated with poor prognosis of prostate cancer, a new strategy for managing treatment of a patient suspected of having indolent prostate cancer is investigated. The method involves assessing prostate cancer in vitro by calculating a concentration of sPLA2-IIA in the patient, wherein a concentration of sPLA2-IIA in the patient that is greater than a cutoff value of sPLA2-IIA (2.0 ng/mL) indicates the presence of prostate cancer. A sample from the patient suspected of having indolent prostate cancer is collected via blood draw. The concentration of sPLA2-IIA in the patient is determined by calculating the concentration of sPLA2-IIA in plasma via ELISA as previously described.

As set forth in Table 13, the patient is treated for prostate cancer where the calculated concentration of sPLA2-IIA is greater than the cutoff value (2.0 ng/mL). However, the patient is placed under active surveillance where the calculated concentration of sPLA2-IIA is less than the cutoff value (2.0 ng/mL). When placed under active surveillance, the patient is monitored by serially calculating the level of sPLA2-IIA to ensure that such concentration remains below the cutoff value.

TABLE 13
Current and Proposed Prostate Cancer Therapy Strategy
Current prostate cancer therapy strategy

| | | | |
|---|---|---|---|
| Aggressive | PSA > 10 ng/ml | → | Therapy |
| cancer | Gleason score 7~10, | | |
| | >50% core biopsy positive | | |
| | stage I | | |
| Indolent | PSA < 10 ng/ml | → | Active surveillance |
| cancer | Gleason score 6 | | |
| | <50% core biopsy positive | | |
| | stage I | | |

Proposed prostate cancer therapy strategy

| | | | |
|---|---|---|---|
| Aggressive | PSA > 10 ng/ml | → | Therapy |
| cancer | Gleason score 7~10, | | |
| | >50% core biopsy positive | | |
| | stage I | | |

| | |
|---|---|
| Indolent cancer | 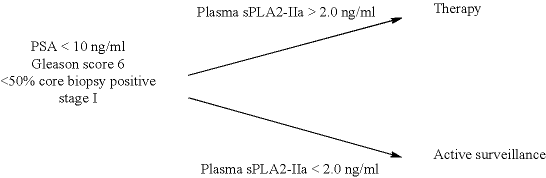 |

Experimental Results.

It is believed that such strategy for managing treatment of a patient may provide a tool for personalized therapy against prostate cancer.

It is noted that terms like “preferably,” “generally,” “commonly,” and “typically” are not utilized herein to limit the scope of the claims or to imply that certain features are critical, essential, or even important to the structure or function of the claims. Rather, these terms are merely intended to highlight alternative or additional features that may or may not be utilized in a particular embodiment of the present disclosure.

For the purposes of describing and defining the present disclosure it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

All documents cited are incorporated herein by reference; the citation of any document is not to be construed as an admission that it is prior art with respect to the present disclosure.

While particular embodiments of the present disclosure have been illustrated and described, it would be obvious to one skilled in the art that various other changes and modifications can be made without departing from the spirit and scope of the disclosure. It is therefore intended to cover in the appended claims all such changes and modifications that are within the scope of this disclosure.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

What is claimed is:

1. A kit for assessing lung cancer in patients with solitary pulmonary nodules, the kit comprising:
reagents for detection and/or quantification of serum secretory phospholipase A₂-IIA in plasma;
reagents for detection and/or quantification of carcinoembryonic antigen in plasma; and
reagents for detection and/or quantification of cytokeratin-19 fragment in plasma.

2. The kit of claim 1, wherein:
the reagents for detection and/or quantification of serum secretory phospholipase A₂-IIA in plasma comprise at least one antibody having specific binding affinity to the serum secretory phospholipase A₂-IIA in the plasma.

3. The kit of claim 1, wherein the reagents for detection and/or quantification of serum secretory phospholipase A₂-IIA in plasma comprise:
a capture antibody having specific binding affinity to the serum secretory phospholipase A₂-IIA in the plasma,
an enzyme conjugated to a detection antibody having specific binding affinity to the serum secretory phospholipase A₂-IIA in the plasma, and
a substrate of the enzyme, wherein action of the enzyme upon the substrate produces at least one detectable product.

4. The kit of claim 3, wherein:
the capture antibody having specific binding affinity to the serum secretory phospholipase A₂-IIA in the plasma is anti-serum secretory phospholipase A₂-IIA,
the enzyme conjugated to the detection antibody having specific binding affinity to the serum secretory phospholipase A₂-IIA in the plasma is serum secretory phospholipase A₂-IIA-acetylcholinesterase-fragment antigen-binding conjugate, and
the substrate is 5,5′-dithiobis-(2-nitrobenzoic acid).

5. The kit of claim 3, wherein:
the reagents for detection and/or quantification of serum secretory phospholipase A₂-IIA in plasma further comprise a wash buffer and a blocking protein.

6. The kit of claim 1, wherein:
the reagents for detection and/or quantification of carcinoembryonic antigen in plasma comprise at least one antibody having specific binding affinity to the carcinoembryonic antigen in the plasma.

7. The kit of claim 1, wherein the reagents for detection and/or quantification of carcinoembryonic antigen in plasma comprise:
a capture antibody having specific binding affinity to the carcinoembryonic antigen in the plasma,
an enzyme conjugated to a detection antibody having specific binding affinity to the carcinoembryonic antigen in the plasma, and
a substrate of the enzyme, wherein action of the enzyme upon the substrate produces at least one detectable product.

8. The kit of claim 7, wherein:
the capture antibody having specific binding affinity to the carcinoembryonic antigen in the plasma is anti-carcinoembryonic antigen,
the enzyme conjugated to the detection antibody having specific binding affinity to the carcinoembryonic antigen in the plasma is horseradish peroxidase conjugated to anti-carcinoembryonic antigen, and
the substrate is 3,3′,5,5′-tetramethylbiphenyl-4,4′-diamine.

9. The kit of claim 7, wherein:
the reagents for detection and/or quantification of carcinoembryonic antigen in plasma further comprise a stopping solution.

10. The kit of claim 1, wherein:
the reagents for detection and/or quantification of cytokeratin-19 fragment in plasma comprise at least one antibody having specific binding affinity to the cytokeratin-19 fragment in the plasma.

11. The kit of claim 1, wherein the reagents for detection and/or quantification of cytokeratin-19 fragment in plasma comprise:
a capture antibody having specific binding affinity to the cytokeratin-19 fragment in the plasma,
an enzyme conjugated to a detection antibody having specific binding affinity to the cytokeratin-19 fragment in the plasma,
a substrate of the enzyme, wherein action of the enzyme upon the substrate produces at least one detectable product, and
a stopping solution, wherein the stopping solution terminates the action of the enzyme upon the substrate.

12. The kit of claim 11, wherein:
the capture antibody having specific binding affinity to the cytokeratin-19 fragment in the plasma is anti-cytokeratin 19 antibody,
the enzyme conjugated to the detection antibody having specific binding affinity to the anti-cytokeratin 19 in the plasma is horseradish peroxidase conjugated to anti-cytokeratin 19 antibody, and
the substrate is 3,3′,5,5′-tetramethylbiphenyl-4,4′-diamine.

13. The kit of claim 1, further comprising:
reagents for detection and/or quantification of squamous cell carcinoma antigen in plasma; and
reagents for detection and/or quantification of neuron specific enolase in plasma.

14. The kit of claim 13, further comprising:
reagents for detection and/or quantification of progastrin-releasing peptide in plasma.

15. A kit for assessing lung cancer in patients with solitary pulmonary nodules, the kit comprising:
reagents for detection and/or quantification of serum secretory phospholipase A₂-IIA in plasma, wherein the reagents for detection and/or quantification of serum secretory phospholipase A₂-IIA in plasma comprise:
a capture antibody having specific binding affinity to the serum secretory phospholipase A₂-IIA in the plasma,
an enzyme conjugated to a detection antibody having specific binding affinity to the serum secretory phospholipase A₂-IIA in the plasma, and
a substrate of the enzyme conjugated to the detection antibody having specific binding affinity to the serum secretory phospholipase A₂-IIA in the plasma, wherein action of the enzyme conjugated to the detection antibody having specific binding affinity to the serum secretory phospholipase A₂-IIA in the plasma upon the substrate produces at least one detectable product;
reagents for detection and/or quantification of carcinoembryonic antigen in plasma, wherein the reagents for detection and/or quantification of carcinoembryonic antigen in plasma comprise:
a capture antibody having specific binding affinity to the carcinoembryonic antigen in the plasma,
an enzyme conjugated to a detection antibody having specific binding affinity to the carcinoembryonic antigen in the plasma, and
a substrate of the enzyme conjugated to the detection antibody having specific binding affinity to the carcinoembryonic antigen in the plasma, wherein action of the enzyme conjugated to the detection antibody having specific binding affinity to the carcinoembryonic antigen in the plasma upon the substrate produces at least one detectable product; and
reagents for detection and/or quantification of cytokeratin-19 fragment in plasma, wherein the reagents for detection and/or quantification of cytokeratin-19 fragment in plasma comprise:
a capture antibody having specific binding affinity to the cytokeratin-19 fragment in the plasma,
an enzyme conjugated to a detection antibody having specific binding affinity to the cytokeratin-19 fragment in the plasma, and
a substrate of the enzyme conjugated to the detection antibody having specific binding affinity to the cytokeratin-19 fragment in the plasma, wherein action of the enzyme conjugated to the detection antibody having specific binding affinity to the cytokeratin-19 fragment in the plasma upon the substrate produces at least one detectable product.

16. The kit of claim 15, further comprising:
reagents for detection and/or quantification of squamous cell carcinoma antigen in plasma; and
reagents for detection and/or quantification of neuron specific enolase in plasma.

17. An in vitro method for assessing lung cancer in a patient comprising utilizing the kit of claim 1.

18. The method of claim 17, wherein the method comprises:
(a) contacting a portion of a sample from the patient with:
the reagents for detection and/or quantification of serum secretory phospholipase A₂-IIA in plasma;
the reagents for detection and/or quantification of carcinoembryonic antigen in plasma; and
the reagents for detection and/or quantification of cytokeratin-19 fragment in plasma;
(b) calculating levels of serum secretory phospholipase A₂-IIA, carcinoembryonic antigen, and cytokeratin-19 fragment based on the contacting in step (a); and
(c) assessing as indicating lung cancer in the patient if the calculated levels of serum secretory phospholipase A₂-IIA, carcinoembryonic antigen, and cytokeratin-19 fragment are elevated relative to cutoff values of serum secretory phospholipase A₂-IIA, carcinoembryonic antigen, and cytokeratin-19.

19. The method of claim 18, wherein:
step (a) further comprises contacting a portion of the sample from the patient with reagents for detection and/or quantification of squamous cell carcinoma antigen and reagents for detection and/or quantification of specific binding agent for neuron specific enolase;
step (b) further comprises calculating levels of squamous cell carcinoma antigen and neuron specific enolase based on the contacting in step (a).

20. An in vitro method for assessing lung cancer in a patient, the method comprising:
(a) contacting a portion of a sample from the patient with:
a specific binding agent for serum secretory phospholipase A₂-IIA,
a specific binding agent for carcinoembryonic antigen, and
a specific binding agent for cytokeratin-19 fragment;
(b) calculating levels of serum secretory phospholipase A₂-IIA, carcinoembryonic antigen, and cytokeratin-19 fragment based on the contacting in step (a); and
(c) assessing as indicating lung cancer in the patient if the calculated levels of serum secretory phospholipase A₂-IIA, carcinoembryonic antigen, and/or cytokeratin-19 fragment are elevated relative to cutoff values of serum secretory phospholipase A₂-IIA, carcinoembryonic antigen, and cytokeratin-19.

21. The method of claim 20, wherein:
step (a) further comprises contacting a portion of the sample from the patient with a specific binding agent for squamous cell carcinoma antigen and a specific binding agent for neuron specific enolase;
step (b) further comprises calculating levels of squamous cell carcinoma antigen and neuron specific enolase based on the contacting in step (a).

22. The method of claim 20, wherein the sample is plasma.

23. The method of claim 20, wherein:
the cutoff value of serum secretory phospholipase A₂-IIA is 2.4 ng/mL,
the cutoff value of carcinoembryonic antigen is 6.0 ng/mL, and
the cutoff value of cytokeratin-19 fragment is 3.3 ng/mL.
